(19) United States
(12) Patent Application Publication
(10) Pub. No.: US 2008/0138430 A1
(43) Pub. Date: Jun. 12, 2008

(54) Temperature-Sensitive Nanoparticles for Controlled Drug Delivery

(76) Inventors: Donald E. Owens, Houston, TX (US); Nicholas A. Peppas, Austin, TX (US)

Correspondence Address: BAKER BOTTS, LLP, 910 LOUISIANA, HOUSTON, TX, 77002-4995, US

(21) Appl. No.: 11/862,753

(22) Filed: Sep. 27, 2007

Related U.S. Application Data
(60) Provisional application No. 60/827,096, filed on Sep. 27, 2006.

(51) Int. Cl. A61K 9/51; A61K 47/32

(52) U.S. Cl. 424/497

(57) ABSTRACT

The present invention generally relates to controlled drug delivery. More specifically, the present invention relates to novel device/system and extracorporeally-controlled method of drug delivery. In some embodiments, the present invention provides a system comprising a thermally-active metal nanoshell; and a temperature-responsive interpenetrating polymer network having at least one therapeutic agent disposed therein; wherein the thermally-active metal nanoshell is proximate to the temperature-responsive interpenetrating polymer network. In some embodiments, the present invention relates to a particle composition comprising a thermally-active metal nanoshell and a temperature-responsive interpenetrating polymer network. A method is also provided comprising: providing a plurality of the particles; and irradiating the particles so as to effect a temperature-induced swelling of the temperature-responsive interpenetrating polymer network.

### CROSS-REFERENCE TO RELATED APPLICATION

This application claims priority to U.S. Provisional Application Ser. No. 60/827,096 filed Sep. 27, 2006, which is incorporated by reference herein.

### STATEMENT OF GOVERNMENT INTEREST

This disclosure was made with support under grant number DGE-0333-080 awarded by the National Science Foundation. The government has certain rights in the invention.

### BACKGROUND

The present invention relates generally to therapeutic drug-delivery systems, and specifically to such systems with particles comprising a metal nanosphere core and a temperature-sensitive interpenetrating network polymer shell in which therapeutic drugs are dispersed and operable for release upon radiation-induced heating of the metal nanosphere.

Research in the field of biomaterials and drug delivery is moving toward individually-tailored intelligent therapeutic systems which are capable of responding to and correcting undesirable conditions, on a molecular level, in the body (Heller, A., “Integrated medical feedback systems for drug delivery.” AIChE J., 51, 1054-1066, 2005; Langer, R. and N. A. Peppas, “Advances in biomaterials, drug delivery, and bionanotechnology.” AIChE J., 49, 2990-3006, 2003). These systems will mimic natural biosystems in their size, structure, and function, and will therefore need to be miniaturized using advanced nanofabrication techniques.

Nanotechnology, or the study of materials with a characteristic length scale on the order of 100 nanometers or less, is a rapidly growing field of research due to the unique properties of materials on this scale. Because of their relatively small size the percentage of surface atoms to bulk atoms is orders of magnitude greater in nanomaterials than in macroscopic materials. Since surface atoms are in a different molecular environment than bulk atoms, unique and unexpected properties can result in nanomaterials as this ratio is increased. A macroscopic sample of material whose properties are principally defined by the properties of bulk atoms can be very different from a nanoscopic sample of the same material whose properties are now dependant on a combination of bulk and surface atom properties. For instance, bulk gold in its natural state displays a yellowish color. However, nanospheres of gold in solution display a range of colors from blue to red and even beyond the visible spectrum depending on their scattering and absorption characteristics, which are in turn related to their size, shape, and other factors that can be affected by the ratio of surface to bulk atoms (Sokolov, K., J. Aaron, B. Hsu, D. Nida, A. Gillenwater, M. Follen, C. MacAulay, K. Adler-Storthz, B. Korgel, M. Descour, R. Pasqualini, W. Arap, W. Lam, and R. Richards-Kortum, “Optical systems for In vivo molecular imaging of cancer.” Technol. Cancer Res. Treat., 2, 491-504, 2003).

The development of nanoscale polymer systems is of interest in modern research as well, because there is the possibility to combine the advantages of traditional macroscale polymer properties, such as swelling, with nanoscale properties, such as lower characteristic diffusion times due to shorter characteristic lengths and localized heating effects. Also, due to the size limitations imposed by the body's natural defense mechanisms, such as the reticuloendothelial system, polymer systems 0.3 μm in diameter and smaller will be required for the development of effective in vivo intelligent therapeutic systems (Stolnik, S., L. Illum, and S. S. Davis, “Long circulating microparticle drug carriers.” Adv. Drug Deliv. Rev., 16, 195-214, 1995; Peracchia, M. T., “Stealth nanoparticles for intravenous administration.” S. T. P. Pharma Sci., 13, 155-161, 2003; Gref, R., Y. Minamitake, M. T. Peracchia, V. Trubetskoy, V. Torchilin, and R. Langer, “Biodegradable long-circulating polymeric nanospheres.” Science, 263, 1600-1603, 1994).

Over the last few years a variety of parenteral synthetic polymer systems (i.e., biodegradable, osmotic, pH responsive, and others) and medical devices (i.e., insulin pumps) have been developed in the hope of achieving intelligent therapeutic function (Heller, A., “Integrated medical feedback systems for drug delivery.” AlChE J., 51, 1054-1066, 2005, Tamada, J. and R. Langer, “The Development Of Polyanhydrides For Drug Delivery Applications.” J. Biomater. Sci.-Polym. Ed., 3, 315-353, 1992; Langer, R., “Drug delivery and targeting.” Nature, 392, 5-10, 1998; Podual, K., F. J. Doyle, and N. A. Peppas, “Glucose-sensitivity of glucose oxidase-containing cationic copolymer hydrogels having poly(ethylene glycol) grafts.” J. Control. Release, 67, 9-17, 2000). To date, however, none of these systems has been able to successfully combine the aspects of molecular recognition, biocompatibility, intelligent response, and non-invasive external therapeutic control.

### FIGURES

Some specific example embodiments of the disclosure may be understood by referring, in part, to the following description and the accompanying drawings.

FIG. 1 is a schematic illustration of a typical stealth nanocomposite particle drawn to scale (gold nanoshell diameter of 40 nm) with a section removed to reveal the inner layers of the composite;

FIG. 2 is a schematic illustration of a poly(acrylic acid)/polyacrylamide IPN where the networks are physically entangled but not covalently bound to one another;

FIG. 3 is a schematic illustration of intermolecular hydrogen bonding in a poly(acrylic acid)/polyacrylamide IPN;

FIG. 4 is a graphical representation of the swelling behavior as a function of temperature of PAA/PAAm IPN and P(AA-co-AAm) random co-polymer disks, where the black arrows indicate the swelling behavior of two gels which have identical monomer, crosslinker, and initiator compositions;

FIG. 5 is an illustration of the hydrogen bonding mechanism that controls swelling in poly(acrylic acid)/polyacrylamide IPNs sometimes referred to as the “Zipper Effect”;

FIG. 6 is an ultrasound image of a simulated cylindrical phantom composed of a mixture of tissue-mimicking gelatin, polystyrene spheres (100 μm) which act as ultrasound scatters, and graphite particles which acts as optical absorbers, where the inclusion in middle of the phantom is created by a high concentration of ultrasound scattering polystyrene spheres in a cylindrical gelatin region surrounded by gelatin which contains a much lower concentration of polystyrene spheres;

FIG. 7 is a photoacoustic image of a simulated phantom composed of a mixture of gelatin, polystyrene spheres, and graphite particles with a laser light source incident from the bottom of the image and transducer array at the top, where the inclusion in middle of the phantom is created by a higher concentration of absorbing graphite particles (10 to 1) in a cylindrical gelatin region surrounded by gelatin which contains a much lower concentration of graphite particles;

FIG. 8 is a combined photoacoustic and ultrasound image of a simulated phantom composed of a mixture of gelatin, polystyrene spheres, and graphite particles with mathematically applied noise filtering to remove noise artifacts;

FIG. 9 is a graph of the linear swelling ratio as a function of temperature of a 1 to 1 AAm/AA IPN in acidic environment, where the scale bars represent one standard deviation, n=3;

FIG. 10 is a representative SEM image of a drop evaporated, gold sputter coated sample of AAm/AA IPN nanoparticles;

FIG. 11 is a representative SEM image of a drop evaporated, gold sputter coated sample of AAm/AA IPN nanoparticles with incorporated gold nanospheres;

FIG. 12 is a representative TEM image of a negatively stained sample of AAm/AA IPN nanoparticles with incorporated gold nanospheres indicated by red arrows; and

FIG. 13 is a representative EDS spectrum of an individual metal-polymer nanocomposite particle obtained using the EDS probe on the JOEL 2010F TEM operating at 200 kV with a probe spot size of 0.5 nm.

FIG. 14 is a representative TEM image of silicon dioxide nanoparticles used to prepare gold nanoshells

FIG. 15 is a representative TEM image of a gold seeded silicon dioxide nanoparticle core which is an intermediate step in the gold nanoshell synthesis method

FIG. 16 is a representative TEM image of gold nanoshells

FIG. 17 is a representative EDS spectrum of an individual gold nanoshell obtained using the EDS probe on the JOEL 2010F TEM operating at 200 kV with a probe spot size of 0.5 nm.

FIG. 18 is a representative TEM image of solid gold nanoparticles

FIG. 19 is a representative low magnification SEM micrograph of polymeric nanoparticles prepared by an inverse microemulsion polymerization and subsequent dialysis and lyophilization. The dried nanoparticles were mounted onto an aluminum SEM stage using double sided carbon tape and gold sputter coated for 30 seconds prior to imaging.

FIG. 20 is a representative high magnification SEM micrograph of polymeric nanoparticles prepared by an inverse microemulsion polymerization and subsequent dialysis and lyophilization. The dried nanoparticles were mounted onto an aluminum SEM stage using double sided carbon tape and gold sputter coated for 30 seconds prior to imaging.

FIG. 21 is a representative dynamic light scattering analysis of the distribution of hydrodynamic diameters present in a sample of polymeric nanoparticles prepared by an inverse microemulsion polymerization after dialysis, lyophilization, and subsequent resuspension for analysis.

FIG. 22 shows the change in the average particle diameter and polydispersity as a function of the emulsifier concentration used in the inverse emulsion polymerization system.

FIG. 23 is a representative SEM micrograph of a discontinuous fragmented polymer film prepared by an inverse microemulsion polymerization with low (˜7 wt %) emulsifier concentration.

FIG. 24 is a representative diagram of the DSC measured heat flow with time for the inverse emulsion polymerization of polyacrylamide. Time zero is set at the point when the reaction begins during the 60° C. isothermal phase of the DSC polymerization analysis.

FIG. 25 is a representative diagram of the rate of polymerization for the inverse emulsion polymerization of a sample of polyacrylamide.

FIG. 26 is a representative diagram of the theoretical and experimental conversion as a function of time for the inverse emulsion polymerization of a sample of polyacrylamide. Time zero is set at the point when the reaction begins during the 60° C. isothermal phase of the DSC polymerization analysis.

FIG. 27 shows DSC analysis of the experimental and theoretical conversion of the various monomer systems utilized in this work. Samples were run in triplicate and the average of all runs ±one standard deviation is recorded. Theoretical values of conversion are not listed for EAA and PAA due to the lack of heat of polymerization values for these materials in the literature.

FIG. 28 is a representative GPC analysis showing the RI trace of a series linear polyacrylamide polymers prepared by an inverse emulsion polymerization technique with varying initiator concentrations.

FIG. 29 shows calculated PEO equivalent molecular weights and polydispersity indices for the various linear polyacrylamide samples examined in this work. The initiator concentration is expressed as a weight percentage of the entire amount of added initiator and monomer. Samples were prepared and tested in triplicate with data showing the average of three measurements ±one standard deviation.

FIG. 30 is a typical PEO standards calibration curve used to calculate the PEO equivalent molecular weight and polydispersity index of the linear polyacrylamide polymers studied in this work.

FIG. 31 shows thermally responsive UCST behavior of a sample of polyacrylamide/poly(acrylic acid) IPN nanoparticles (0.1 mol % crosslinked) suspended in an aqueous pH 3 buffer solution. Error bars represent one standard deviation, n=10.

FIG. 32 shows the effect of crosslinker concentration on the thermally-responsive swelling properties of a series IPN nanoparticle samples, suspended in an aqueous pH 3 buffer solution. Error bars represent one standard deviation, n=10.

FIG. 33 shows The monomer ratios and molar percentages of crosslinker used to prepare the IPN nanoparticles examined in FIG. 32. The maximum relative swelling volume (RSV) ±one standard deviation (STD), n=10, achieved by each system is also listed. The molar ratios of AAm to AA repeat units in the final IPN structure were calculated using the feed ratio of each monomer and their experimentally determined percentage conversion.

FIG. 34 shows the monomer ratios and molar percentages of crosslinker used to prepare the IPN nanoparticles examined in FIGS. 35-38. The maximum relative swelling volume (RSV) ±one standard deviation (STD), n=10, achieved by each system is also listed. The molar ratios of AAm to AA repeat units in the final IPN structure were calculated using the feed ratio of each monomer and their experimentally determined percentage conversion.

FIG. 35 shows the trend in the RSV verses pH that was observed for PAAm/PAA IPN nanoparticles. As expected the initial deprotonation and swelling of the nanoparticles began in the pH range of 4-5, which corresponds well with the literature value of 4.8 for the pKa of PAA. Error bars represent one standard deviation, n=10.

FIG. 36 shows the trend in the RSV versus pH that was observed for PAAm/PMAA IPN nanoparticles. As expected the initial deprotonation and swelling of the nanoparticles began in the pH range of 5-6, which corresponds well with the literature value of 6.15 for the pKa of PAA. Error bars represent one standard deviation, n=10.

FIG. 37 shows the trend in the RSV versus pH that was observed for PAAm/PEAA IPN nanoparticles. As expected the initial deprotonation and swelling of the nanoparticles began in the pH range of 6-7, which corresponds well with the literature value of 7.2 for the pKa of PAA. Error bars represent one standard deviation, n=10.

FIG. 38 shows the trend in the RSV versus pH that was observed for PAAm/PPAA IPN nanoparticles. As expected the initial deprotonation and swelling of the nanoparticles was shifted to a higher pH in the range of 8-9. Error bars represent one standard deviation, n=10.

FIG. 39 shows thermally responsive UCST behavior of a sample of PAAm/PPAA IPN nanoparticles (1 mol % crosslinked) suspended in phosphate buffered saline at a pH of 7.4 and an ionic strength of 150 mM. Error bars represent one standard deviation, n=10. Error bars represent one standard deviation, n=10.

FIG. 40 shows the monomer ratios and molar percentages of crosslinker used to prepare the IPN nanoparticles examined in FIG. 41. The maximum relative swelling volume (RSV) ±one standard deviation (STD), n=10, achieved by each system is also listed. The molar ratios of AAm to AA repeat units in the final IPN structure were calculated using the feed ratio of each monomer and their experimentally determined percentage conversion.

FIG. 41 shows a DLS study of the thermally responsive swelling properties of homopolymer nanoparticles of both polyacrylamide and poly(acrylic acid), a random copolymer of polyacrylamide-co-poly(acrylic acid), and a polyacrylamide/poly(acrylic acid) IPN, suspended in a pH=3 aqueous buffer. Error bars represent one standard deviation, n=10.

FIG. 42 is a representative Zeta potential analysis of a sample of PAAm/PAA IPN nanoparticles showing a negative surface charge, due to the ionization of carboxylic acid groups present in the poly(acrylic acid) portion of the IPN structure, of −19.16±3.89 mV (n=10).

FIG. 43 is a representative Zeta potential analysis of PEG surface grafted PAAm/PAA IPN nanoparticles showing an approximately neutral to slightly positive surface charge of 2.77±1.08 mV (n=10).

FIG. 44 is a FT-IR absorption spectrum of polyacrylamide (PAAm) showing the characteristic absorption bands of this material located at approximately 3360, 3210, 2945, 1665, 1455, 1420, 1345, 1330, and 1117 cm⁻¹.

FIG. 45 is a FT-IR absorption spectrum of poly(acrylic acid) (PAA) showing the characteristic absorption bands of this material located at approximately 3450, 3200, 2960, 2635, 1720, 1456, 1415, 1255, and 1180 cm⁻¹.

FIG. 46 is a FT-IR absorption spectrum of PAAm/PAA IPN nanoparticles before PEGylation showing the characteristic absorption bands of this material located at approximately 3430, 3215, 2950, 1680, 1455, 1416, 1284, and 1180 cm⁻¹.

FIG. 47 is a FT-IR absorption spectrum of PAAm/PAA IPN nanoparticles after PEGylation showing the characteristic absorption bands of this material located at approximately 3410, 3210, 2895, 1675, 1590, 1456, 1413, 1343, 1282, and 1115 cm⁻¹.

FIG. 48 is an SEM micrograph of dried and gold sputter coated metal-polymer nanocomposite particles which clearly illustrates their spherical morphology.

FIG. 49 is a dynamic light scattering analysis of the distribution of hydrodynamic diameters present in a sample of as-prepared metal-polymer nanocomposite particles.

FIG. 50 is a low magnification TEM micrograph of metal-polymer nanocomposite particles. Red arrows indicate the presence of solid gold nanoparticles encapsulated inside larger polymer nanoparticles. Dried buffer salt crystals are also present in the image.

FIG. 51 is a high magnification TEM micrograph of an individual metal-polymer nanocomposite particle. The smaller darker circle is a solid gold nanoparticle and the surrounding lighter circle is a polymeric particle.

FIG. 52 is a representative EDS spectrograph analysis of an individual metal-polymer nanocomposite particle. A portion of the carbon peak and all of the copper peaks in the spectrograph are due to the carbon coated TEM grid on which the sample is mounted, the remainder of the carbon peak as well as the oxygen peak are due to the polymer portion of the nanocomposite particle, and the gold peak is due to the gold particle encapsulated inside the nanocomposite.

FIG. 53 shows the atomic composition of an individual metal-polymer nanocomposite particle as determined by single particle EDS analysis. A small part of the carbon signal and all of the copper signal in the spectrograph are due to the carbon coated TEM grid on which the sample is mounted, the remainder of the carbon signal as well as all of the oxygen signal are due to the polymer portion of the nanocomposite particle, and the gold peak is due to the gold particle encapsulated inside of the nanocomposite.

FIG. 54 is a representative Zeta potential analysis of a sample of metal-polymer nanocomposite particles showing a negative surface charge, due to the ionization of carboxylic acid groups present in the poly(acrylic acid) portion of the IPN, of −23.54±4.15 mV (n=10).

FIG. 55 is a representative Zeta potential analysis of PEG surface grafted gold-polymer nanocomposite particles showing an approximately neutral surface charge of 3.01±1.32 mV (n=10).

FIG. 56 shows the results of the effect of an external laser source on the measured hydrodynamic diameter of a sample of blank IPN nanoparticles. Vertical lines at the minute and 20 minute time point indicate the activation and deactivation of the external laser source, respectively.

FIG. 57 shows the results of the effect of an external laser source on the measured hydrodynamic diameter of a sample of as-prepared metal-polymer nanocomposite particles. Vertical lines at the 10 minute and 20 minute time point indicate the activation and deactivation of the external laser source, respectively.

FIG. 58 is a schematic illustration of the experimental setup used to photoacoustically image a sample of metal-polymer nanocomposite particles.

FIG. 59 is a standard ultrasound image of the dialysis tube used to contain the aqueous particle suspension during imaging. A yellow circle is used to represent the location of the dialysis tubing whose long axis is oriented into the plane of the image. The white area indicates detected ultrasound signal that was produced by sound waves reflecting back to the transducer from the top and bottom of the dialysis tubing.

FIG. 60 is a photoacoustic image of a blank sample of pure ddH₂O used to determine the amount of photoacoustic signal produced by the absorption of laser light by the dialysis tubing without the presence of nanocomposite particles. Bright spots in the image indicate the presence and intensity of the photoacoustic signal while dark blue represents a lack of signal.

FIG. 61 is a photoacoustic image of a sample of metal-polymer nanocomposite particles created by the excitation of the particles with an external 532 nm laser source. Bright spots in the image indicate the presence and intensity of the photoacoustic signal while dark blue represents a lack of signal.

FIG. 62 shows a comparison of the photoacoustic signal intensity down the center of the dialysis tubing for both the blank ddH₂O sample and the metal-polymer nanocomposite sample, clearly indicating a large increase in signal intensity for the metal-polymer nanocomposite sample.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### SUMMARY

The present invention is directed to a novel device/system and extracorporeally-controlled method of drug delivery. In some embodiments, devices are prepared using interpenetrating polymer network (IPN) nanoparticles to create a temperature-sensitive drug delivery device that can respond to extracorporeal triggering mechanisms by swelling in response to increases in temperature and releasing its therapeutic content. The disclosed method requires the incorporation of metal nanoshells inside the IPN, which strongly absorb near infrared (IR) light and convert that light energy to heat. Near IR light (wavelength=800-1200 nm), such as that produced by a Nd:Yag laser, can pass easily and harmlessly through the body.

Unlike typical temperature-controlled release devices/systems, such as those utilizing poly(N-isopropylacrylamide) (PNIPAAm) polymers that shrink in response to increases in temperature, the IPN devices of the present invention swell in response to increases in temperature. As used herein, the term “swell” and its derivatives (e.g. “swelling” and “swollen”) refer to an increase in volume, whereas the term “shrink” and its derivatives (e.g. “shrinking” and “shrunk”) refer to a decrease in volume. This makes them ideal controlled release devices because they can remain in the collapsed (or off) state until activated thermally by the use of a near IR laser source. Also, these particles are of an ideal size, 200-300 nm in diameter, for use as an injectable drug delivery system.

The materials used in the devices/systems and methods of the present invention have the ability to safely localize and release therapeutic levels of potent drugs, such as chemotherapy agents, which would lead to lower systemic doses, reduced side effects, higher patient compliance, and improved quality of life for patients. Additionally, the devices of the present invention represent an in vivo method of drug delivery that is extracorporeally controlled, unlike current technologies which either passively release drug or require an internal signal to activate and release drug. The advantage of this device is that the doctor, patient, relative, or primary health care giver in charge of treatment decisions has the ability to modify or even alter the course of the therapy when necessary.

Under certain conditions it is possible that high temperatures generated inside the nanoparticles might damage or denature proteonic drugs within the IPN matrix. This will generally not be a problem with more stable small molecule drugs, but for proteins this problem can be ameliorated by the careful control of the temperature at which the IPN device transitions into the activated drug delivery state. Additionally, these nanoparticles could also be used in any system where external or temperature controlled volume transition is important, such as values or actuators in micro-fluidic systems.

The features and advantages of the present disclosure will be readily apparent to those skilled in the art upon a reading of the description of exemplary embodiments, which follows.

### DESCRIPTION

In the following description, specific details are set forth such as specific quantities, sizes, etc. so as to provide a thorough understanding of embodiments of the present invention. However, it will be apparent to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

Generally speaking, the present invention is directed to an externally-triggered therapeutic system, the system comprising metal-polymer nanocomposite particles themselves comprising: (a) a thermally-active metal nanoshell; (b) a temperature-responsive interpenetrating polymer network disposed as a shell about the metal nanoshell; and (c) at least one therapeutic agent dispersed throughout the interpenetrating polymer network.

In some embodiments, the present invention is directed to a method comprising: providing a plurality of the metal-polymer nanocomposite particles described above; and irradiating the particles so as to effect a temperature-induced swelling of the temperature-responsive interpenetrating polymer network.

Interpenetrating Polymer Networks

Interpenetrating polymer networks or IPNs were chosen as an exemplary polymer carrier for use in the above-mentioned metal-polymer nanocomposites for several reasons. First, IPNs are able to exhibit a relatively sharp transition with temperature without requiring the use of highly ordered block-copolymers or polymers with very monodisperse molecular weights, which are both typically expensive and difficult to synthesize. Second, IPNs that form secondary hydrogen bonding complexes are also one of the few polymer systems that exhibit a positive sigmoidal swelling transition with temperature (Katono, H., A. Maruyama, K. Sanui, N. Ogata, T. Okano, and Y. Sakurai, “Thermoresponsive Swelling And Drug Release Switching Of Interpenetrating Polymer Networks Composed Of Poly (Acrylamide-Co-Butyl Methacrylate) And Poly (Acrylic-Acid).” J. Control. Release, 16, 215-227, 1991). Finally, IPN synthesis techniques are also extremely flexible and can theoretically be utilized to synthesize IPNs composed of any number of different monomers and crosslinkers in a wide variety of combinations to achieve optimum response characteristics.

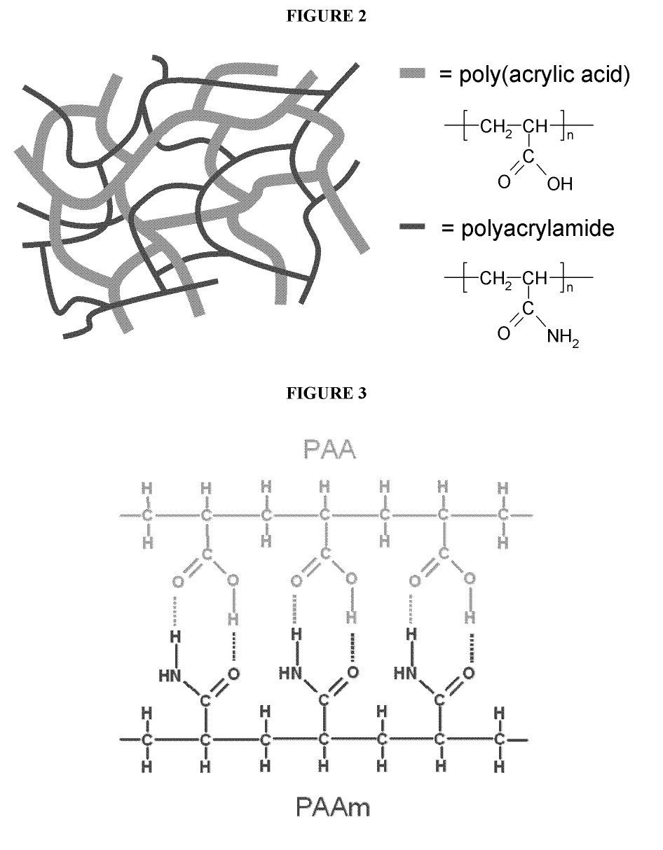

IPNs exhibit these desirable characteristics because of their unique chemical structure, which is generally described as a polymer matrix system that is composed of two independently crosslinked networks that are interpenetrating with one another, but are not covalently bound (Athawale, W. D., S. L. Kolekar, and S. S. Raut, “Recent developments in polyurethanes and poly(acrylates) interpenetrating polymer networks.” J. Macromol. Sci.-Polym. Rev, C43, 1-26, 2003). More specifically, these two independent networks can be any type of polymer system, or even the same polymer as is the case of homo-IPN systems. Different methods can also be used to create the IPN system including sequential-, simultaneous-, latex-, and gradient-IPNs (Chen, L. and S. Chen, “Latex interpenetrating networks based on polyurethane, polyacrylate and epoxy resin.” Prog. Org. Coat., 49, 252-258, 2004). Herein, Applicants have focused on a polyacrylamide/poly(acrylic acid) (PAA/PAAc) latex-IPN, as shown graphically in FIG. 2.

Latex IPNs are typically synthesized by emulsion polymerization of the second monomer together with the crosslinker and activator inside the original seed latex of the first polymer (Athawale, W. D., S. L. Kolekar, and S. S. Raut, “Recent developments in polyurethanes and poly(acrylates) interpenetrating polymer networks.” J. Macromol. Sci.-Polym. Rev, C43, 1-26, 2003). This allows for greater control of the particle size, morphology, and final size distribution than other methods, such as solution/dispersion polymerizations or crushing and sieving methods.

Temperature-sensitivity in IPNs may be achieved in one of two ways. First, standard hydrophobic/hydrophilic interactions such as those attributed to poly(N-isopropyl acrylamide) (PNIPAAm) can be used to create a polymer system that is immiscible in water at higher temperatures and miscible at lower temperatures. Depending on the degree of polydispersity in the molecular weight of the polymer system, this change in miscibility can occur gradually over a broad temperature range (i.e., an exponential or linear response for polydisperse molecular weights) or nearly instantaneously over a narrow temperature range (i.e. a sigmoidal response for monodisperse and block-copolymers). In the latter case, this change in miscibility occurs at what is known as the lower critical solution temperature or LCST of the material. PNIPAAm in an aqueous environment is a well characterized system that exhibits this type of transition at around 32° C. (Zhang, J. and N. A. Peppas, “Morphology of poly(methacrylic acid)/poly(N-isopropyl acrylamide) interpenetrating polymeric networks.” J. Biomater. Sci.-Polym. Ed., 13, 511-525, 2002).

On the other hand, the exact opposite change in miscibility with temperature can also be seen in certain polymer-solvent systems whereby a polymer is immiscible at temperatures below a certain upper critical solution temperature (UCST) and miscible above. Although at first glance these transitions in polymer-solvent miscibility with temperature for UCST and LCST polymers seem similar with only opposite miscibility, the mechanisms that cause this behavior can be quite different.

From a thermodynamic standpoint, when the Gibbs free energy of mixing, ΔGₘᵢₓ, is negative at a given temperature and pressure, mixing is spontaneous and will result in one homogenous phase hence the polymer is miscible with the solvent. However, if ΔGₘᵢₓ is positive then the polymer will be immiscible with the solvent and two phases, namely the polymer phase and the solvent phase will co-exist. From the Gibbs free energy equation of mixing, ΔGₘᵢₓ=ΔHₘᵢₓ−TΔSₘᵢₓ, one can immediately determine that for LCST polymers, ΔHₘᵢₓ and ΔSₘᵢₓ must be negative so that as temperature increases, the second term in the equation eventually cancels out and then increase beyond the value of the first term changing the overall ΔGₘᵢₓ of the polymer-solvent system from an initial negative value corresponding to a miscible system to a positive value corresponding to an immiscible two phase system. This also makes sense from a physical standpoint when one considers a polymer that exhibits LCST behavior such as PNIPAAm. At temperatures below its LCST, PNIPAAm is hydrophilic in nature and miscible with water. As temperature is increased PNIPAAm becomes increasingly hydrophobic in nature and this causes polymer chains free in solution to associate more readily with themselves rather than the aqueous solvent, which leads to the development of two separated pure phases. This separation from a homogenous mixture into two pure components represents a decrease in the entropy of mixing and hence a positive increase in ΔGₘᵢₓ. The main underlying mechanism driving this transformation is the hydrophobic/hydrophilic nature of PNIPAAm.

Since crosslinked networks such as IPNs can never fully dissolve, miscibility with a solvent manifests itself in the form of a swollen gel for miscible states and a collapsed solvent free polymer for immiscible states. For UCST polymer systems it is evident that both ΔHₘᵢₓ and ΔSₘᵢₓ are positive so that as temperature is increased, the exact opposite behavior of LCST materials is observed. However, for the case of PAA/PAAm IPNs, the mechanism that gives these polymer systems their unique UCST behavior is very different from that of PNIPAAm and is based on the presence of secondary intermolecular hydrogen bonding complexes. FIG. 3 graphically illustrates the intermolecular hydrogen bonding complexes that can form between PAA and PAAm polymer changes in an IPN polymer of these materials.

Hydrogen bonds, because of their relatively weak interactions, are stronger at lower temperatures and their strength decreases with increasing temperature (Okano, T., “Molecular Design Of Temperature-Responsive Polymers As Intelligent Materials.” Adv. Polym. Sci., 110, 179-197, 1993). Therefore, at lower temperatures the hydrogen bonding complexes between polymer chains within the IPN are the dominate force and the polymer remains in a collapsed and dry state. However, as temperature is increased above the UCST temperature these bonds become weaker and the hydrophilic nature of the PAA and PAAm polymer dominates, which leads to a rapid hydration and swelling of the particles. This change from an ordered and collapsed immiscible state to a swollen miscible state clearly represents an increase in the entropy of mixing and hence a decrease in ΔGₘᵢₓ to a negative value or spontaneous mixing.

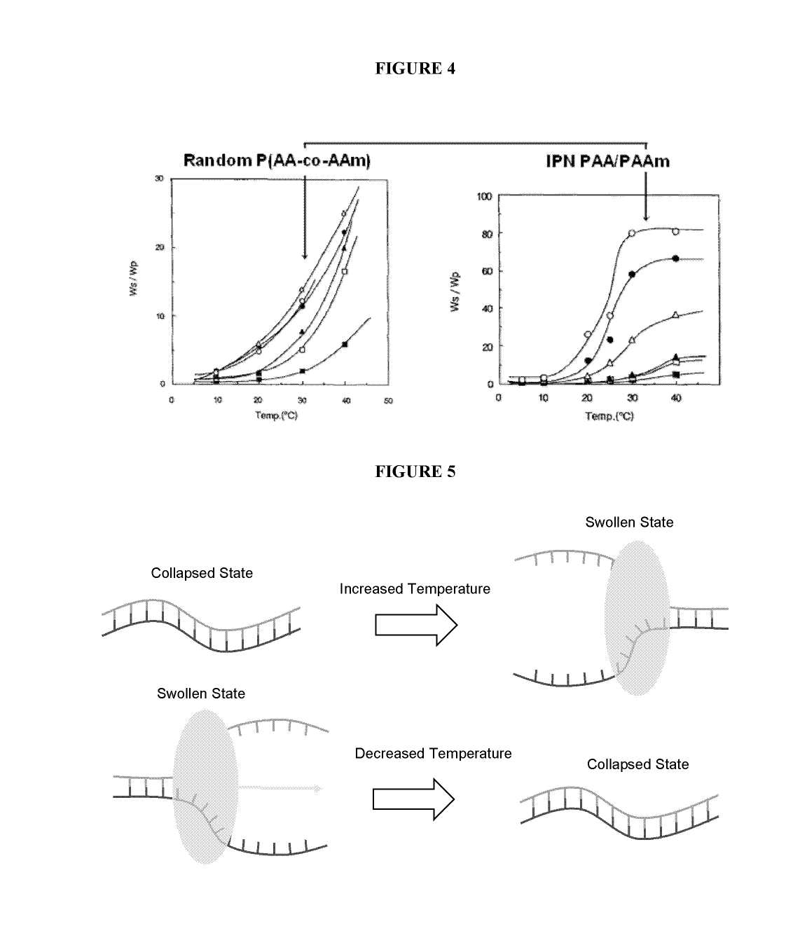

This rapid swelling effect, termed the “zipper effect” by Okano and associates (Katono, H., A. Maruyama, K. Sanui, N. Ogata, T. Okano, and Y. Sakurai, “Thermoresponsive Swelling And Drug Release Switching Of Interpenetrating Polymer Networks Composed Of Poly (Acrylamide-Co-Butyl Methacrylate) And Poly (Acrylic-Acid).” J. Control. Release, 16, 215-227, 1991; Okano, T., “Molecular Design Of Temperature-Responsive Polymers As Intelligent Materials.” Adv. Polym. Sci., 110, 179-197, 1993; Aoki, T., M. Kawashima, H. Katono, K. Sanui, N. Ogata, T. Okano, and Y. Sakurai, “Temperature-Responsive Interpenetrating Polymer Networks Constructed With Poly(Acrylic Acid) And Poly(N,N-Dimethylacrylamide).” Macromolecules, 27, 947-952, 1994), is due to the long-range hydrogen bonding order that occurs in IPN structures as opposed to standard random co-polymers. Proof of this order can also be seen in comparison studies like the one shown in FIG. 4, between IPN and random co-polymers where the two polymers are created with the same monomer, crosslinker, and initiator compositions (Katono, H., A. Maruyama, K. Sanui, N. Ogata, T. Okano, and Y. Sakurai, “Thermoresponsive Swelling And Drug Release Switching Of Interpenetrating Polymer Networks Composed Of Poly (Acrylamide-Co-Butyl Methacrylate)

And Poly (Acrylic-Acid).” J. Control. Release, 16, 215-227, 1991). From this study, it is clear that the IPN polymer exhibits a sigmoidal swelling response where as the random co-polymer exhibits a more exponential response. Under certain conditions this effect can also be reversed as illustrated in FIG. 5.

Metal Nanoshells

In order to control the swelling and release of encapsulated molecules and compounds from the intelligent therapeutic systems externally, a localized heating source within the polymer nanoparticle itself is needed. However, in order to be effective in vivo the heating source has to meet several important requirements. First, it must be small enough to fit inside the intelligent therapeutic particle without making the entire system larger than 300 nm in diameter. It must also heat via a safe non-invasive external trigger that is capable of reaching the nanoshell at high penetration depths in vivo. Finally, it should also be able to act as a contrast agent for imaging as well. Therefore, metal nanoshells were chosen because of their compliance with these guidelines.

The chemistry, optical properties, and physical characteristics of metal nanoshells make them unique and important materials for use in the field of nanotechnology. Metal nanoshells typically comprise a spherical core of dielectric material (such as SiO₂ or Au₂S), which is surrounded by a thin layer of conducting metal such as gold. The properties of these nanoshells can be well characterized by Mie theory which is based on a rigorous solution to Maxwell's equations in spherical coordinates with boundary conditions appropriate for a sphere. Mie scattering theory requires that the dielectric function of the particle and embedding medium be specified. Therefore, for solid gold particles the optical properties of the system can be fully described by specifying the nanoparticle radius and using the bulk frequency dependent dielectric constant ∈(ω). However, for core shell particles, changes in the dielectric constant throughout the particle must be taken into account by the use of a position dependent dielectric function ∈(ω,r). Applying Mie scattering theory with this new position-dependant dielectric function reveals the exciting result that the plasmon absorption peak location depends only upon the ratio of the shell thickness to the total radius. Therefore, for a given shell thickness the larger the total radius of the particle the further the peak is red shifted, and in the limit of an infinitely thick shell the absorption tends toward that of a solid gold particle at approximately 520 nm. This result suggests that metal nanoshells can be tuned to absorb a specific wavelength of electromagnetic radiation over a wide range of wavelength by simply controlling the size of the particle and thickness of its shell.

Experimental preparations of non-semiconductor colloidal metal nanoshells in the literature typically involve the use of one of two methods. The first reaction scheme of this type was described in the literature in 1994 for the synthesis of gold-gold sulfur nanoshells (Zhou, H. S., I. Honma, H. Komiyama, and J. W. Haus, “Controlled Synthesis And Quantum-Size Effect In Gold-Coated Nanoparticles.” Phys. Rev. B. 50, 12052-12056, 1994). In this method, equal volumes of aqueous solutions of 2 mM gold chloride (HAuCl₄) and 1 mM sodium sulfide (Na₂S) are added together at room temperature under vigorous stirring. This results in the spontaneous nucleation and growth of solid Au₂S nanospheres, which quickly plateau at approximately 40 nm in diameter. It was purposed by Zhou et al. (Zhou, H. S., I. Honma, H. Komiyama, and J. W. Haus, “Controlled Synthesis And Quantum-Size Effect In Gold-Coated Nanoparticles.” Phys. Rev. B. 50, 12052-12056, 1994) that after the formation of these particles a slow process of diffusion begins to occur whereby S²⁻ ions present in solution begin to reduce the surface layers of the Au₂S solid particles creating a solid gold shell. If left undisturbed, this process will go to completion whereby all solid Au₂S particles are converted to solid pure gold particles. This process, however, can be stopped at anytime by the addition of a capping agent such as a methoxy poly(ethylene glycol) thiol (mPEG-SH).

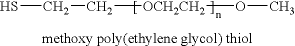

Therefore, the desired optical properties of the colloidal suspension can be selected by monitoring the absorption peak of the solution during the reaction and adding the capping agent when the absorption peak is centered at the desired frequency. The main disadvantage of this reaction scheme is that it does not allow for much variation of the final particle size, which is typically around 40 nm in diameter. This reaction scheme also does not allow for control of the core material. However, the main advantage of this reaction is that it does create relatively small (<50 nm) monodisperse tunable nanoshells.

The second reaction scheme was first described in the literature in 1998 for the synthesis of gold-silicon dioxide nanoshells (Oldenburg, S. J., R. D. Averitt, S. L. Westcott, and N. J. Halas, “Nanoengineering of optical resonances.” Chem. Phys. Lett., 288, 243-247, 1998). This reaction scheme involves a layer-by-layer approach to creating nanoshells and is much more experimentally complex. Briefly, a suspension of solid silica particles of a desired diameter are synthesized using the Stöber or other relevant processes (Stöber, W., A. Fink, and E. Bohn, “Controlled growth of monodisperse silica spheres in the micron size range.” J. Colloid and Interface Sci., 26, 62-69, 1968). The surface of these particles, which are inherently functionalized with OH groups, are then reacted with 3-aminopropyltriethoxysilane (APTES), which covalently bonds to the particles via a condensation reaction between the oxysilane groups of APTES and the OH groups on the surface of the SiO₂ particle.

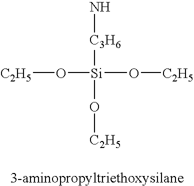

The final result of this step in the reaction scheme is the synthesis of an amine functionalized surface on the silica particle. These amine functionalized silica particles are then placed in solution with 2 nm gold seeds. The gold seeds then associate with and bond to the amine groups on the surface of the silica particles to create a gold seed decorated silica particle. The final step in this reaction scheme is to place these seeded gold silica particles in solution with aqueous gold which is then reduced onto the surface of the gold seeds with a reducing agent such as formaldehyde. As the aqueous gold deposits on the gold seeds they grow in size until they are large enough to touch and coalesce into a solid continuous shell. Once this shell is completed absorption effects like the ones seen in the gold-gold sulfur nanoshells begin to occur as the thickness of the gold shell increase. Once again this reaction can be stopped once the desired absorption peak is reached by the addition of a capping agent such as mPEG-SH.

The advantages of this reaction are that, in theory, any dielectric core material that can be functionalized with amine groups could be used, such as a biodegradable polymer. Also the final size of the particle can be controlled to some extend by choosing the size of the core. The main disadvantages of this reaction are its experimental complexity and numerous stages. It is also hard to make very small sized tunable nanoshells (<50 nm) using this technique due to the difficulty in producing well defined monodisperse silica or other cores this size and the minimum thickness (4-5 nm) required to create a complete gold shell layer verses the very small shell to core ratio that is required to achieve good near infrared absorption.

Photoacoustic Imaging

Because in some embodiments the intelligent therapeutic systems will need to be imaged at relatively high depths in vivo an imaging modality that is capable of high tissue penetration depths is needed. Photoacoustic imaging utilizes a nanosecond pulsed laser and ultrasound transducer, which are both capable of achieving high tissue penetration depths, when a near infrared pulsed laser is used, to create an image. Also since the nanoshells are already tuned to absorb NIR light they are well suited for use as contrast agents in photoacoustic imaging.

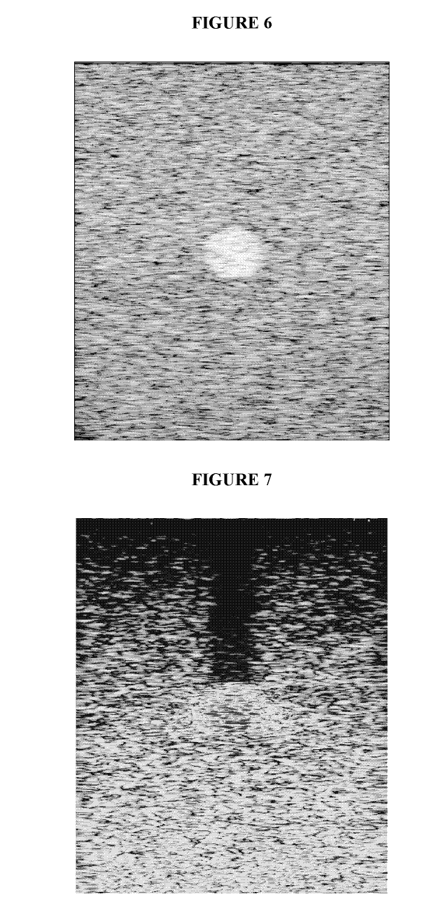

Photoacoustic imaging, also known as optoacoustic or thermoacoustic imaging, is a technique that utilizes electromagnetic radiation and acoustic waves (sound) to image tissues and other compounds similar to the way that ultrasound images tissues. In ultrasound imaging sound waves are produced by a transducer and propagated into an area of tissue at a specific time with a set frequency and amplitude. These waves then interact with compounds and tissue and are reflected back to the transducer which measures the distance they travel (time of flight) and any change in frequency or amplitude that occurs. This information is then translated into an image like the one shown in FIG. 6 of a tissue-mimicking gelatin phantom with 100 μm polystyrene spheres added as ultrasound contrast agents.

On the other hand, in the case of photoacoustic imaging a transducer is again used to measure the time of flight, frequency, and amplitude of sound waves, however, these sound waves are generated internally by the interaction of electromagnetic radiation with optically absorbing compounds inside the tissue rather than produced externally by a transducer. More specifically, an area of interest is irradiated with a nanosecond pulse of low energy laser light. This pulse will typically be 5-10 ns long due to the stress confinement criteria, (tₚ<<d/νₛ where tₚ=pulse duration, d=diameter of irradiated volume, and νₛ speed of sound in the medium) which requires a pulse duration much shorter than the stress relaxation time of the irradiated volume to produce ultrasonic acoustic waves (Jacques, S. L., “Role Of Tissue Optics And Pulse Duration On Tissue Effects During High-Power Laser Irradiation.” Appl. Optics, 32, 2447-2454, 1993; Jacques, S. L., “Laser Tissue Interactions—Photochemical, Photothermal, And Photomechanical.” Surg. Clin.-North Am., 72, 531-558, 1992). This light energy is then absorbed and dissipated via thermoelastic expansion, which in turn produces broadband ultrasonic acoustic waves (Wang, Y. W., X. Y. Xie, X. D. Wang, G. Ku, K. L. Gill, D. P. O'Neal, G. Stoica, and L. V. Wang, “Photoacoustic tomography of a nanoshell contrast agent in the in vivo rat brain.” Nano Lett., 4, 1689-1692, 2004). It is these waves that are then detected and used to form an image of the irradiated volume of interest like the one shown in FIG. 7 of the same tissue-mimicking gelatin phantom in FIG. 6 with graphite particles added as photoacoustic contrast agents.

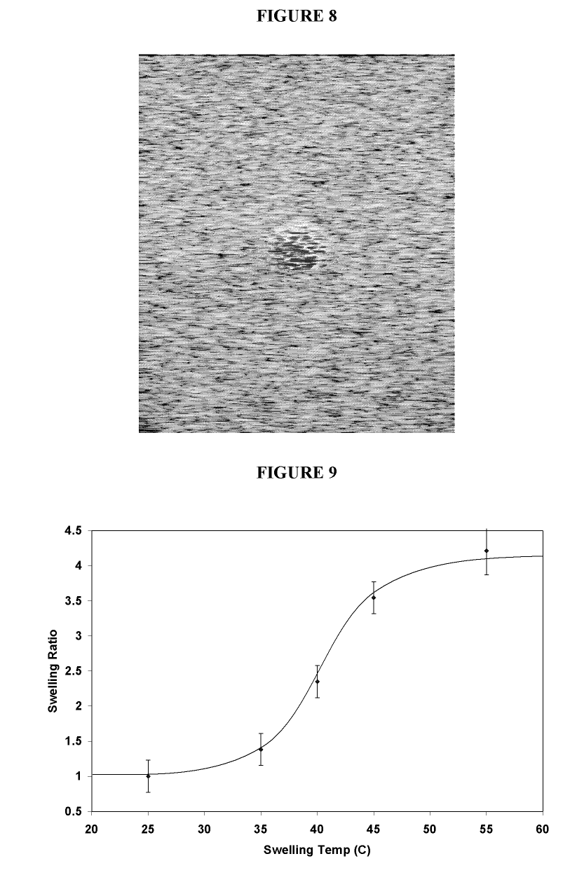

This type of imaging is ideal for injectable metal-polymer nanocomposite systems for several reasons. First, laser light in the range of 800-1000 nm or the near infrared region, which is a form of non-ionizing radiation that is also capable of high penetration depths in vivo, can be used to initiate thermoelastic expansion. This type of imaging is also inherently noninvasive. Furthermore, the laser fluence level that will be utilized for imaging (5-10 mJ/cm²) is 3-5 times lower than the safe level of laser irradiation for this wavelength of light as defined by the American National Standards and the FDA. Also, this wavelength of light is the same that will be used to heat the nanocomposites so that the same laser set to different modes (i.e., pulsed and continuous wave) might be able to perform both imaging and therapeutic tasks. Since a transducer will already be in place to measure the signal produced by optically absorbing compounds it would also be possible to collect standard ultrasound images as well. This is beneficial because ultrasound imaging can provide detailed structural information at high penetration depths while photoacoustic imaging will add contrast and functional information. Finally, an example of the types of images that can be produced using this combined ultrasound and photoacoustic imaging approach is shown in FIG. 8. In this case, the ultrasound image of the tissue-mimicking phantom, FIG. 6, and the photoacoustic image of the same tissue-mimicking phantom, FIG. 7, were combined together with mathematical filtering to remove noise artifacts and produce a high contrast structural and functional image of the tissue-mimicking phantom.

Temperature-Sensitive Nanoparticles for Controlled Drug Delivery

Generally, methods of the present invention involve (a) a synthesis and characterization of novel temperature-responsive interpenetrating polymer network nanoparticles for use in an intelligent therapeutic system capable of loading and releasing therapeutic agents in response to controlled temperature fluctuations; (b) the incorporation into the IPN nanoparticles of thermally-active particles such as metal nanoshells to act as both a control mechanism and contrast agent for the overall intelligent therapeutic system; and (c) an understanding of the swelling, controlled release, and imaging capabilities of these intelligent therapeutic systems via near infrared laser activation in aqueous environment.

In some embodiments, a properly-formulated polyacrylamide/poly(acrylic acid) IPN nanosphere with incorporated metal nanoshell core can be controlled externally in vivo, via a near infrared laser, leading to a novel, non-invasive, locally confined, intelligent therapeutic and imaging system. Also, with the addition of either grafted or surface adsorbed poly(ethylene glycol) (PEG) chains, these particles can become stealth or long-circulating intelligent therapeutic systems.

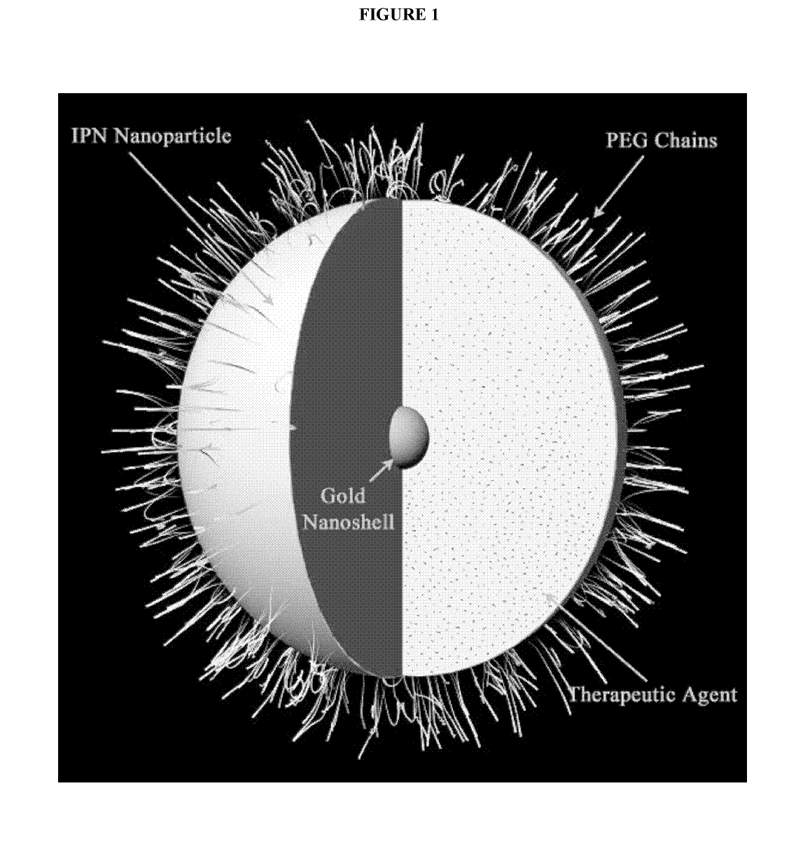

The metal-polymer intelligent therapeutic systems described herein comprise a collection of individual particles that each contains several components or layers which are physically and chemically bound together to create a novel intelligent and responsive metal-polymer nanocomposite. The nanocomposite particles described herein are composed of metal nanoshell cores surrounded by temperature-sensitive polymers which encapsulate desired molecules or compounds and are functionalized on their surface with a stealth agent such as poly(ethylene glycol). A schematic illustration of a single nanocomposite particle is shown in FIG. 1, where a section has been cut away to reveal the inner layers of the particle.

The first and outer-most layer of such an above-mentioned composite nanoparticle is the poly(ethylene glycol) or surface PEG layer. PEG is a well known polymer that is utilized frequently in medical devices due to its high biocompatibility and unique properties (Plard, J. P. and D. Bazile, “Comparison of the safety profiles of PLA(50) and Me.PEG-PLA(50) nanoparticles after single dose intravenous administration to rat.” Colloid Surf B-Biointerfaces, 16, 173-183, 1999). One of these properties is its ability to convey stealth characteristics to materials to which it has been attached (Peracchia, M., S. Harnisch, H. Pinto-Alphandary, A. Gulik, J. Dedieu, D. Desmaele, J. d'Angelo, R. Muller, and P. Couvreur, “Visualization of in vitro protein-rejecting properties of PEGylated stealth (R) polycyanoacrylate nanoparticles.” Biomaterials, 20, 1269-1275, 1999; Bazile, D., C. Prudhomme, M. T. Bassoullet, M. Marlard, G. Spenlehauer, and M. Veillard, “Stealth Me.PEG-PLA nanoparticles avoid uptake by the mononuclear phagocytes system.” J. Pharm. Sci., 84, 493-498, 1995; Storm, G., S. O. Belliot, T. Daemen, and D. D. Lasic, “Surface modification of nanoparticles to oppose uptake by the mononuclear phagocyte system.” Adv. Drug Deliv. Rev., 17, 31-48, 1995). This process, which is commonly referred to as PEGylation, involves either the adsorbing or grafting of PEG chains to a material's surface. PEGylation allows materials in the form of polymeric nanoparticles, that would normally be filter out of the blood immediately after injection to remain in circulation for hours and sometimes even days (Gref, R., Y. Minamitake, M. T. Peracchia, V. Trubetskoy, V. Torchilin, and R. Langer, “Biodegradable long-circulating polymeric nanospheres.” Science, 263, 1600-1603, 1994; Bazile, D., C. Prudhomme, M. T. Bassoullet, M. Marlard, G. Spenlehauer, and M. Veillard, “Stealth Me. PEG-PLA nanoparticles avoid uptake by the mononuclear phagocytes system.” J. Pharm. Sci., 84, 493-498, 1995; Stolnik, S., B. Daudali, A. Arien, J. Whetstone, C. R. Heald, M. C. Garnett, S. S. Davis, and L. Illum, “The effect of surface coverage and conformation of poly(ethylene oxide) (PEO) chains of poloxamer 407 on the biological fate of model colloidal drug carriers.” BBA-Biomembranes, 1514, 261-279, 2001). The longer these nanoparticles remain in circulation the more chances they have to interact with tissues and undesirable components in the body.

In the more specific case of cancer treatment, PEGylation allows a polymeric material to take advantage of the characteristic leaky vasculature of cancer. Due to the rapid and irregular growth of tumor cells, large intercellular openings can form—leading to what are known as leaky sites. For example, in MCa-IV mouse carcinomas these openings can be as large as 1.7 μm (mean diameter) with sizes ranging anywhere from 0.3-4.7 μm (Hashizume, H., P. Baluk, S. Morikawa, J. W. McLean, G. Thurston, S. Roberge, R. K. Jain, and D. M. McDonald, “Openings between defective endothelial cells explain tumor vessel leakiness.” Am. J. Pathol., 156, 1363-1380, 2000). By comparison, normal endothelial fenestrae are typically less than 50 nm in diameter (Bearer, E. L., L. Orci, and P. Sors, “Endothelial Fenestral Diaphragms—A Quick-Freeze, Deep-Etch Study.” J. Cell Biol., 100, 418-428, 1985). Long circulating PEGylated or “stealth” nanoparticles have increased accumulation in tumors because of their preferential extravasation via this leaky vasculature. Finally, a small percentage of surface PEG chains could also be functionalized with antibodies, peptides, or other ligands to achieve active targeting of integrins, growth factors, and receptors that are upregulated in tumors (Valtola, R., P. Salven, P. Heikkila, J. Taipale, H. Joensuu, M. Rehn, T. Pihlajaniemi, H. Weich, R. deWaal, and K. Alitalo, “VEGFR-3 and its ligand VEGF-C are associated with angiogenesis in breast cancer.” Am. J. Pathol., 154, 1381-1390, 1999; Holash, J., P. C. Maisonpierre, D. Compton, P. Boland, C. R. Alexander, D. Zagzag, G. D. Yancopoulos, and S. J. Wiegand, “Vessel cooption, regression, and growth in tumors mediated by angiopoietins and VEGF.” Science, 284, 1994-1998, 1999; Cox, G., J. L. Jones, R. A. Walker, W. P. Steward, and K. J. O'Byrne, “Angiogenesis and non-small cell lung cancer.” Lung Cancer, 27, 81-100, 2000).

The next component or layer in the particles of such intelligent therapeutic systems is the temperature-sensitive polymer shell. This polymer shell, which surrounds the metal nanoshell core of the intelligent therapeutic system, gives the system its intelligent response characteristics and encapsulates desired molecules or compounds that are to be released. The polymer shell is typically 80 nm in thickness and composed of two or more independent polymer networks that are not chemically bonded to one another, but are interpenetrating such that they can not be separated. Together, these networks form what is termed an interpenetrating polymer network or IPN. Any polymer network or even copolymer networks can be used to form an IPN, but Applicants have initially focused on polymer networks composed of individual polyacrylamide (PAAm) and poly(acrylic acid) (PAA) networks (FIG. 2).

Latex IPNs of PAAm and PAA are well known for their ability to swell rapidly in response to small increases in temperature and to shrink or collapse rapidly in response to small decreases in temperature (Katono, H., A. Maruyama, K. Sanui, N. Ogata, T. Okano, and Y. Sakurai, “Thermoresponsive Swelling And Drug Release Switching Of Interpenetrating Polymer Networks Composed Of Poly (Acrylamide-Co-Butyl Methacrylate) And Poly (Acrylic-Acid).” J. Control. Release, 16, 215-227, 1991). It is this on/off (swollen/collapsed) behavior and positive swelling behavior with temperature that makes them ideal intelligent responsive materials for controlled release. Also, due to their small size and lower characteristic diffusion time, drug can be released from these systems in a matter of seconds as compared to standard pills or large particles which can take hours even up to days to achieve complete release once they are activated.

The third and final component of such above-described intelligent therapeutic systems is the metal nanoshell particle located at the core of the nanocomposite particle, which acts as both the control mechanism or trigger for IPN swelling and hence release, and also as a photoacoustic contrast agent. These nanoshells are typically 40 nm in diameter and are comprised of a dielectric 34 nm diameter core that is surrounded by a roughly 3 nm thick metal shell. Nanoshells, in general, can be tuned to absorb a specific wavelength of electromagnetic radiation anywhere from the visible region (500 nm) up to the infrared region (3000 nm) (Oldenburg, S. J., R. D. Averitt, S. L. Westcott, and N. J. Halas, “Nanoengineering of optical resonances.” Chem. Phys. Lett., 288, 243-247, 1998).

In some embodiments of the present invention, the intelligent therapeutic systems utilize gold nanoshells that are optimized to absorb light in the near infrared region at around 808 nm. This wavelength of electromagnetic radiation is preferred for intelligent therapeutic systems because it is non-ionizing and capable of penetrating deeply inside the human body (>10 cm in breast tissue, 7 cm in muscle tissue, and 4 cm in skull/brain tissue) with minimal attenuation so that intelligent therapeutic systems located inside the body can still be reached with this method (Weissleder, R., “A clearer vision for in vivo imaging.” Nat. Biotechnol., 19, 316-317, 2001). Once the light energy has been absorbed by the nanoshells it is converted into thermal energy that is transmitted locally to the surrounding IPN nanosphere. This thermal energy then heats the IPN causing it swell and release any agent that has been entrapped or encapsulated inside the collapsed polymer matrix. Because this heating effect only occurs within the irradiated volume, release can be selectively activated inside a tumor or other tissue of interest and thus reduce or eliminate systemic or overall dosing of the body. Also, if a rapidly pulsed laser is used to delivery the near infrared light then this absorbed energy can be used to trigger thermoelastic expansion, which in turn produces broadband ultrasonic acoustic waves that can then be used for imaging.

Synthesis and Characterization of Interpenetrating Polymer Network Nanoparticles

In some embodiments, latex-IPNs composed of polyacrylamide, poly(acrylic acid), and co-polymers of the two are synthesized using a thermally-initiated free-radical inverse emulsion polymerization. Particular emphasis is placed on both the molar ratio of AAm to AA in the initial reaction mixture and the ratio of AAm to AA repeat units in the final latex-IPN based on measurements of conversion with the hypothesis that a 1 to 1 molar ratio of AAm to AA repeat units in the final IPN will lead to the largest and sharpest swelling transition in acidic conditions (pH<3). This hypothesis is based on investigations of macroscopic sized AAm/AA IPN polymer discs (diameter>2 mm) which showed optimum performance at a 1 to 1 molar ratio of repeat units in the final polymer structure (Katono, H., A. Maruyama, K. Sanui, N. Ogata, T. Okano, and Y. Sakurai, “Thermoresponsive Swelling And Drug Release Switching Of Interpenetrating Polymer Networks Composed Of Poly (Acrylamide-Co-Butyl Methacrylate) And Poly (Acrylic-Acid).” J. Control. Release, 16, 215-227, 1991; Okano, T., “Molecular Design Of Temperature-Responsive Polymers As Intelligent Materials.” Adv. Polym. Sci., 110, 179-197, 1993; Aoki, T., M. Kawashima, H. Katono, K. Sanui, N. Ogata, T. Okano, and Y. Sakurai, “Temperature-Responsive Interpenetrating Polymer Networks Constructed With Poly(Acrylic Acid) And Poly(N,N-Dimethylacrylamide).” Macromolecules, 27, 947-952, 1994).

Due to the low pKa of poly(acrylic acid) (pKa˜4.5), at neutral pH the carboxylic acid group of this polymer will be virtually completely deprotonated and will therefore, not be able to participate in hydrogen bonding (Bouillot, P. and B. Vincent, “A comparison of the swelling behaviour of copolymer and interpenetrating network microgel particles.” Colloid Polym. Sci., 278, 74-79, 2000). Therefore, the swelling behavior with temperature, from 15° C. to 55° C. in increments of 5° C., of homo-IPNs of polyacrylamide and co-polymer IPNs of polyacrylamide with small amounts of poly(acrylic acid) (0-10 mol %) should be investigated in neutral pH conditions.

Reactions can be conducted in a three-necked round bottom flask equipped with a condenser, nitrogen purge, and inlet feed. Hexane is the inverse emulsion continuous phase, acrylamide (AAm) or acrylamide (AAm) and acrylic acid (AA), the monomers, N,N′-methylenebisacrylamide (MBAAm), the crosslinker, bis(2-ethylhexyl) sulfosuccinate sodium salt (AOT) and polyethylene glycol laurylether (Brij 30), the emulsifiers, and ammonium persulfate (APS) and N,N,N′,N′-tetramethylethylenediamine (TEMED), the initiator and accelerator, respectively.

In a typical AAm/AA IPN synthesis, AOT and Brij 30 were added to a round bottom flask and dissolved in hexane under magnetic stirring. Separately in a 30 ml vial, glacial AAm, MBAAm, deionized distilled water (ddH₂O), and APS were combined and sonicated to ensure a homogenous mixture (Bouillot, P. and B. Vincent, “A comparison of the swelling behaviour of copolymer and interpenetrating network microgel particles.” Colloid Polym. Sci., 278, 74-79, 2000; Daubresse, C., C. Grandfils, R. Jerome, and P. Teyssie, “Enzyme immobilization in reactive nanoparticles produced by inverse microemulsion polymerization.” Colloid Polym. Sci., 274, 482-489, 1996). This mixture was then added to the round bottom flask containing hexane and dissolved emulsifiers under vigorous stirring and the resulting inverse emulsion was purged for 30 minutes with nitrogen. At the completion of the purge, TEMED was injected into the system to initiate the free radical polymerization. This polymerization was then allowed to proceed for 2 hours at room temperature to completion. At the end of this period the reaction mixture was immersed in an ice bath and opened to the atmosphere. A second 30 ml vial was then charged with glacial AA, MBAAm, ddH₂O, and APS and sonicated as before. This mixture was then added to the same round bottom flask containing the now fully crosslinked PAAm latex nanoparticles. As before this mixture was again purged with nitrogen for 30 minutes followed by the injection of TEMED and reaction for 2 hours at room temperature (Okano, T., “Molecular Design Of Temperature-Responsive Polymers As Intelligent Materials.” Adv. Polym. Sci., 110, 179-197, 1993; Bouillot, P. and B. Vincent, “A comparison of the swelling behaviour of copolymer and interpenetrating network microgel particles.” Colloid Polym. Sci., 278, 74-79, 2000). The final latex IPN particles were then recovered by hexane removal at reduced pressure and elevated temperature (29.5 in Hg, 40° C.), and emulsifier removal with repeated ethanol washes and centrifugation. For a homo-IPN synthesize the steps above would be preformed exactly the same way only with AAm monomers added in both the first and second aqueous reaction mixtures. Finally for a co-polymer IPN varying ratios of AAm and AA monomer would be added together in one or both of the aqueous reaction mixtures.

Characterization of the swelling properties, and more specifically the hydrodynamic diameter, of the final IPN materials will be performed in both acidic and aqueous environments using a Brookhaven ZetaPlus Dynamic Light Scattering instrument. This instrument measures the scattering of light from particles in suspension under diffusive Brownian motion. The constructive and destructive interference of this randomly scattered light results in an average intensity of scattered light at a fixed angle (typically 90° with respect to the incident light beam) with superimposed fluctuations. The decay times of these fluctuations are then related to the diffusion constant and therefore the hydrodynamic radius of the particles by the Stokes-Einstein equation shown below. Small particles moving rapidly cause faster decaying fluctuations than large particles moving more slowly. In this equation, D is the translational diffusion coefficient of the particle, kB is Boltzmann's constant, T is the temperature in ° K., η(t) is the temperature

$$D = \frac{{k}_{B} T}{3 π η ( t ) d}$$

dependant viscosity of the suspension medium, and d is the hydrodynamic diameter of the particle.

IPNs of AAm and AA (1 to 1 initial molar ratio) have already been successfully synthesized and characterized in acidic (pH=3) buffered water using the Brookhaven DLS. These nanoparticles exhibited a sigmoidal swelling response with temperature at around 40±5° C. with a linear swelling ratio increase of greater than four times the collapsed particle diameter (˜200 nm) as shown in FIG. 9.

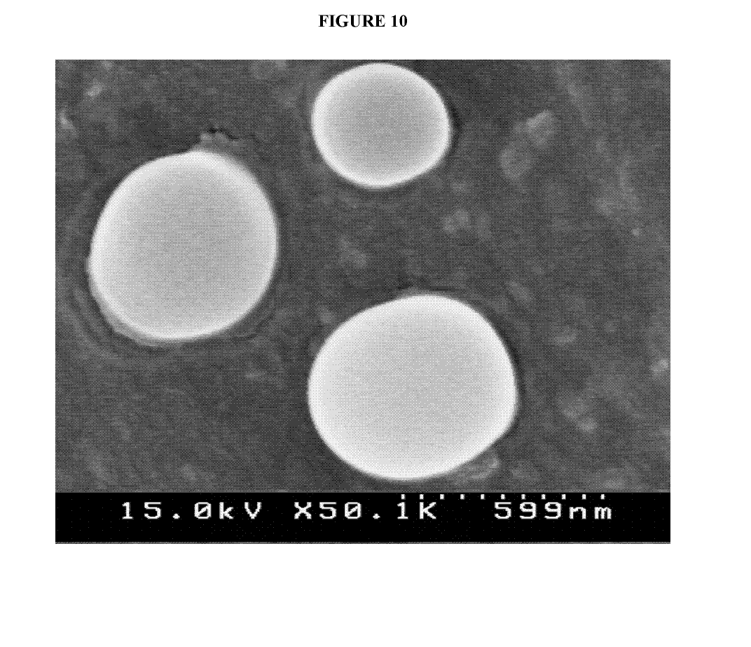

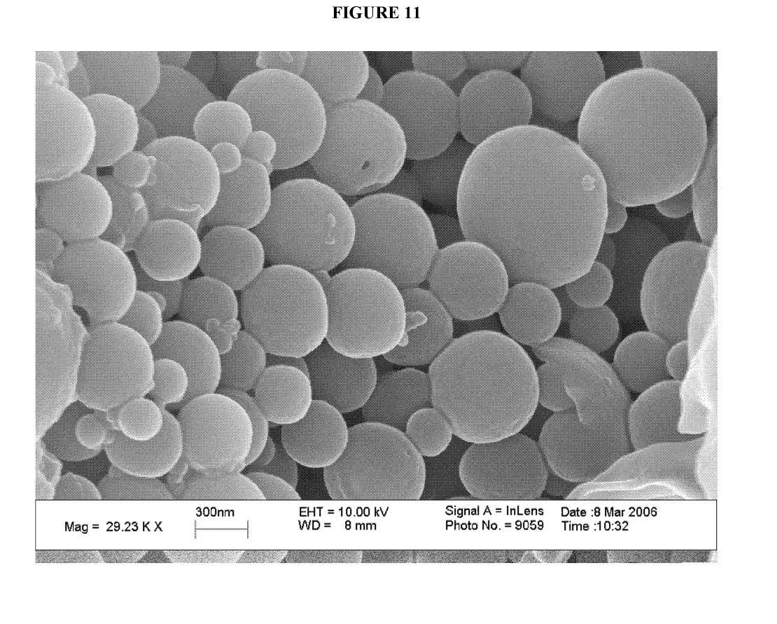

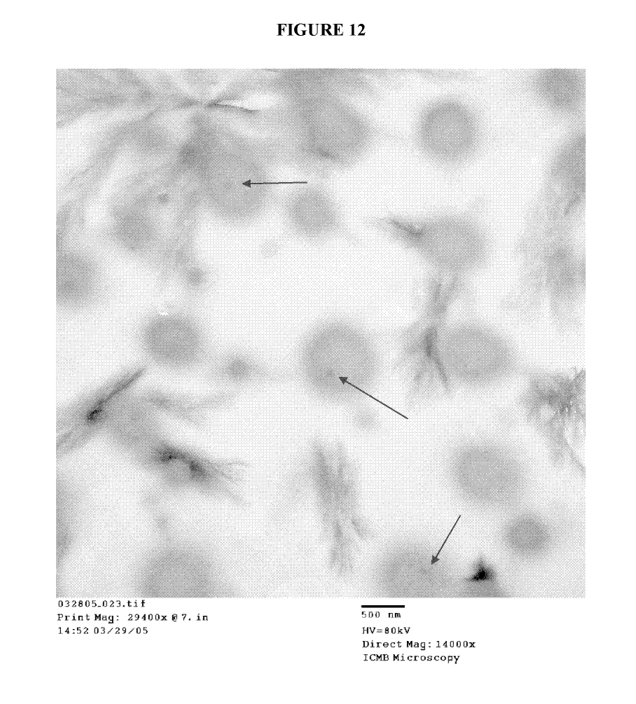

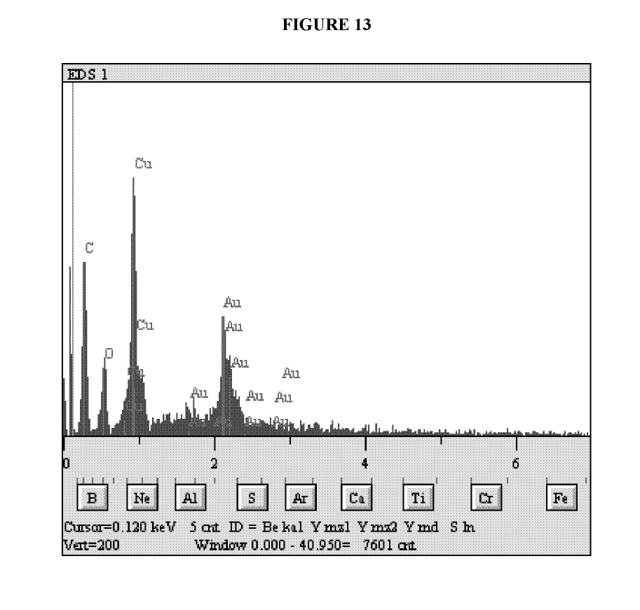

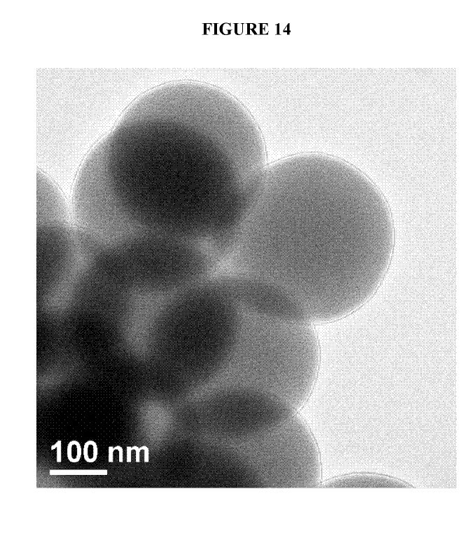

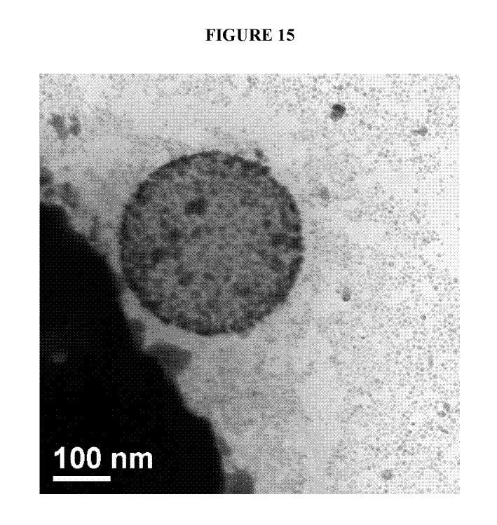

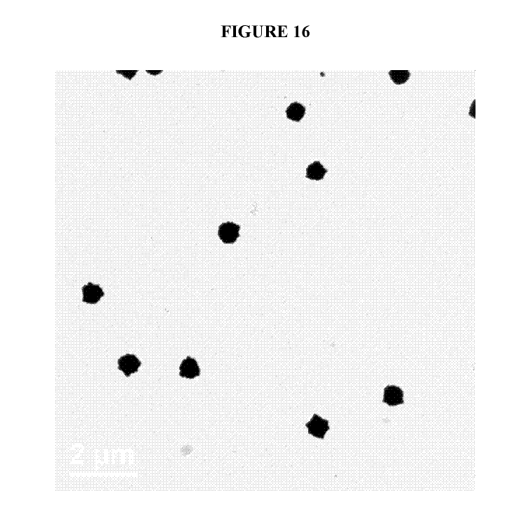

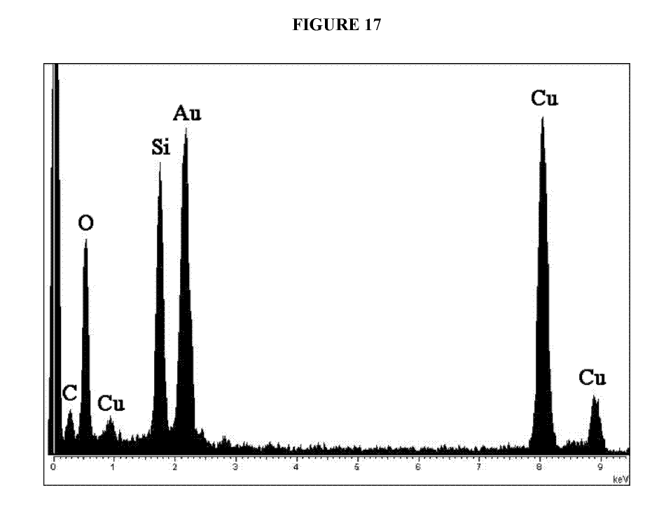

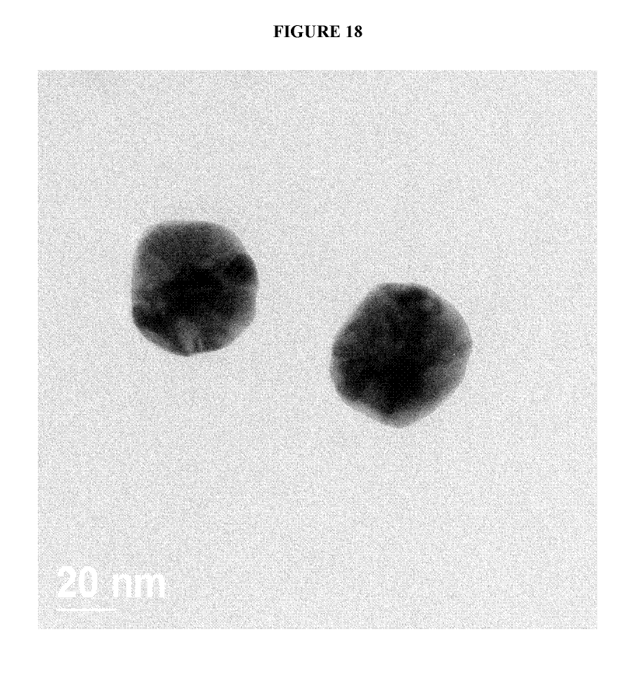

The spherical morphology of these particles has also been confirmed using a LEO Model 1530 scanning electron microscope (SEM). Samples were prepared by drop evaporation overnight on aluminum stages followed by gold sputter coating for 30 seconds using a Pelco Model 3 sputter-coater. A representative image of these particles is shown in FIG. 10.

Finally measurements of the percent conversion and kinetics of both the AAm and AA polymerization steps in the IPN synthesis will be examined using either peak area analysis of the rate of disappearance of the carbonyl double bond at 1710 cm⁻¹ for the acrylic acid monomer and 1610 cm⁻¹ for the acrylamide with a Thermo Mattson Infinity Gold FT-IR spectrophotometer equipped with a Pike Technologies multiple reflection HATR with heated trough plate and liquid flow-through cell and/or monitoring of the heat of polymerization using a Mettler Toledo RC1 reaction calorimeter.

Synthesis and Characterization of Intelligent Therapeutic Systems

In some embodiments, gold nanoshells can be prepared using the synthesis techniques described above and in the literature (e.g., Zhou, H. S., I. Honma, H. Komiyama, and J. W. Haus, “Controlled Synthesis And Quantum-Size Effect In Gold-Coated Nanoparticles.” Phys. Rev. B. 50, 12052-12056, 1994; Oldenburg, S. J., R. D. Averitt, S. L. Westcott, and N. J. Halas, “Nanoengineering of optical resonances.” Chem. Phys. Lett., 288, 243-247, 1998; Oldenburg, S. J., R. D. Averitt, and N. J. Halas, U.S. Pat. No. 6,685,986, Feb. 3, 2004; Loo, C., A. Lin, L. Hirsch, M. H. Lee, J. Barton, N. Halas, J. West, and R. Drezek, “Nanoshell-enabled photonics-based imaging and therapy of cancer.” Technol. Cancer Res. Treat., 3, 33-40, 2004). These nanoshells can then be incorporated into the latex-IPN polymers via in situ polymerization of the latex emulsion with gold nanoshells present in the aqueous phase. Due to their relatively large size, compared to the monomers, crosslinkers, imitators, and other small molecules present in the reaction mixture, these nanospheres should be added directly to the continuous phase with a small amount of water prior to the addition of the emulsifiers to ensure that upon emulsification these nanoshells will be present in the emulsified aqueous phase. The success of this in situ polymerization will be explored using both scanning electron microscopy (SEM) to probe the final morphology of the intelligent therapeutic systems and transmission electron microscopy (TEM) to examine the location and efficiency of nanoshell encapsulation.

Exemplary techniques utilizing latex-IPN inverse emulsion polymerizations with 40 nm diameter solid gold nanospheres in place of gold nanoshells (which are more costly and difficult to produce) have been conducted by Applicants. SEM and TEM results with batches that contained high concentrations of gold spheres (˜10¹⁷ spheres/m³ in the aqueous phase) compared to the theoretical concentration of monomer droplets in an emulsion (˜10¹⁴ droplets/m³) lead to a large variation in particles size and morphology as seen in SEM images (FIG. 11). The size and morphology differences of these particles are due to a large variation in the number (˜10-1000) of gold spheres that are contained in each individual polymer sphere as evidenced in TEM images (FIG. 12). Further proof of the presence of gold inside the latex-IPN particles was confirmed using an Oxford INCA Energy Dispersive Spectroscopy (EDS) probe on the JEOL 2010F TEM operating at 200 kV and a probe spot size of 0.5 nm to examine individual metal-polymer nanocomposite particles. The results of analysis, shown in FIG. 12 and Table 1, clearly indicate the presence of gold, carbon, and oxygen atoms in the sample. The additional copper peak that is present in the EDS spectrum originates from the placement of the sample on a copper TEM grid for imaging. Therefore, in order control and optimize the incorporation of gold nanoparticles inside the latex-IPN particles, kinetic studies of the particle formation mechanism both with and without gold nanoparticles present will be conducted using either FT-IR spectroscopy and/or reaction calorimetry.

While not intending to be bound by theory, particle formation in inverse emulsion polymerizations is hypothesized to occur through one of three mechanisms depending on the concentration and type of emulsifiers used. These mechanisms include homogenous, micellar, and droplet nucleation. Due to the high concentration of emulsifiers used in this polymerization (>10 wt %) it is likely that the predominate mechanism of latex-IPN (without gold nanoshells present) particle formation is micellar in nature (El-Aasser, M. S. and E. D. Sudol, Eds. Emulsion Polymerization and Emulsion Polymers. John Wiley and Sons: New York. 1997). However, polymerizations in the presence of gold nanospheres, which are large (120-40 nm diameter) relative to the size of the micelles (5-10 nm diameter), most likely shift the particle formation mechanism to a combination of micellar and droplet nucleation. This in turn can lead to a polydisperse or bimodal particle size distribution with larger particles that contain gold nanoparticles and smaller ones that do not.

Shifting this polymerization mechanism from a combination of micellar and droplet nucleation to one of predominantly droplet nucleation can be achieved in several ways. Standard methods include varying the concentration and type of emulsifiers and initiators used in the polymerization as well as the amount of sheer or mechanical agitation applied to the emulsion (El-Aasser, M. S. and E. D. Sudol, Eds. Emulsion Polymerization and Emulsion Polymers. John Wiley and Sons: New York. 1997). Accordingly, one approach of ensuring droplet nucleation using the systems of the present disclosure, is the local heating of gold nanoparticles within the droplets to initiate polymerization rather than the addition of TEMED accelerator at room temperature. Regardless of the method used, elucidation and control of the particle formation mechanism results in enhanced control over the final nanocomposite particle size, morphology, and gold nanoshell encapsulation ratio.

Once these nanoshells are successfully incorporated into latex-IPN particles in a controlled manner, examination of laser-induced swelling in aqueous environment can be carried out using an 808 nm continuous wave NIR diode laser coupled with a Brookhaven ZetaPlus DLS instrument to study the swelling properties of these intelligent therapeutic systems.

To examine the photoacoustic properties of these intelligent therapeutic systems, a Q-switched Nd:YAG laser operating at 808 nm with a 5 ns pulse rate coupled with 7.5 MHz ultrasound transducer array can be utilized. The intelligent therapeutic nanoparticles can be embedded in a cylindrical tissue-mimicking gelatin phantom along with 100 μm polystyrene spheres to enhance ultrasound scattering. These phantoms can then be imaged using photoacoustic techniques against a background of tissue-mimicking gelatin without embedded intelligent therapeutic nanoparticles.

### EXAMPLES

IPN Particle Synthesis

Materials

Acrylic acid (AA, inhibited with 200 ppm hydroquinone monomethyl ether), methacrylic acid (MAA, inhibited with 250 ppm hydroquinone monomethyl ether), 2-ethylacrylic acid (EAA, inhibited with 150 ppm butylated hydroxytoluene), 2-propylacrylic acid (PAA, inhibited with 150 ppm butylated hydroxytoluene), N,N′-methylenebisacrylamide (MBAAm), polyethylene glycol laurylether (Brij 30), cyclohexane, and sodium bis(2-ethylhexyl) sulfosuccinate (AOT) were obtained from Sigma Aldrich (Milwaukee, Wis.), acrylamide (AAm) and ammonium persulfate (APS) were obtained from Fisher Scientific (Hampton, N.H.), and acryl-poly(ethylene glycol)-N-hydroxysuccinimide (MW=5,000) was obtained from Nektar Therapeutics (San Carlos, Calif.). All materials were used as received.

Synthesis of Polymeric Nanoparticles

PAAm/P(AA, MAA, EAA, and PAA) IPN polymer nanoparticles were synthesized by a two stage sequential inverse emulsion polymerization method. Unless otherwise stated, the inverse emulsion solution consisted of an 81 wt % cyclohexane continuous phase, with a 13 wt % surfactant phase (AOT and Brij 30 in a 2:1 ratio), and a 6 wt % aqueous phase. The exact composition of the aqueous phase was varied depending on the type of monomer system used and the final polymer structure that was desired. However, a typical aqueous phase consisted of approximately 11.7 wt % monomer, 2 wt % crosslinker, 5.3 wt % initiator, and 81 wt % water.

In a standard experiment, a 3-neck round bottom flask equipped with a condenser, nitrogen purge line, and overhead mechanical stirrer was first charged with the entire volume of cyclohexane to be used in the polymerization. To this the entire emulsifier phase was added and dissolved under vigorous stirring. For the first stage of the sequential IPN polymerization one-half of the total aqueous phase was added containing only the acrylamide monomer along with crosslinker, initiator, and deionized distilled water (ddH₂O). This mixture was then purged with nitrogen gas for 30 minutes to remove oxygen and homogenized (Ultra-Turrax T25, IKA, Wilmington, N.C.) at 24,000 rpm for 5 minutes.

After homogenization, the polymerization was then initiated thermally by immersion of the reaction vessel in a 60° C. bath and allowed to react to completion (typically 2 hours). Upon completion of the first stage of the IPN synthesis, the second stage was then started by adding the other half of the aqueous phase, consisting of additional crosslinker, initiator, and the second monomer to be used in the IPN (AA, MAA, EAA, or PAA), to the same 3-neck round bottom flask as before. The vessel was again purged with nitrogen gas, homogenized, and allowed to react at 60° C. for two hours, thus resulting in the formation of the final PAAm/PAA IPN nanoparticles.

PAAm and PAA crosslinked homopolymer nanoparticles, P(AAm-co-AA) crosslinked copolymer nanoparticles, and linear PAAm polymer chains were all also made using the same inverse emulsion polymerization system as the IPN particles except that the aqueous phases were combined and added in just one step. In the case of the copolymer nanoparticles, both aqueous phases (containing both AAm and AA monomers) were polymerized together in one step. In the case of the homopolymer nanoparticles, both phases were again combined, but only contained AAm monomer in the case of PAAm homopolymer nanoparticles and AA monomer in the case of the PAA nanoparticles. In the case of the linear polymer chains, monomer and initiator were added together and polymerized as usual in the absence of crosslinker.

All of the various polymer batches were then collected and purified by removal of the cyclohexane phase with elevated temperature and reduced pressure (40° C./50 mmHg) in a rotary evaporator (RE-121, Buchi, Flawil, Switzerland). This was followed by precipitation of the particles or linear polymer chains out of the emulsifier phase with the addition of excess ethanol and subsequent pelting and washing (three times) by centrifugation (Centra CL3R, Thermo IEC, Waltham, Mass.) at 3200 rcf for 60 minutes. The purified polymer pellet was then resuspended in deionized water in preparation for dialysis cleaning, lyophilization, and/or PEGylation depending on the experimental requirements.

Polymeric nanoparticles were also PEGylated to increase their biocompatibility and colloidal stability. For these experiments, the resuspended polymer nanoparticles were PEGylated using standard N-hydroxysuccinimide (NHS) chemistry to covalently bind linear PEG chains to the primary amine groups of the polyacrylamide portion of the IPN (24). Typically, the pH of an aqueous suspension of polymer nanoparticles, at a concentration of approximately 1 mg of polymer per ml of ddH₂O, was raised to the range of 7.5-8.5. A heterofunctional acryl-poly(ethylene glycol)-NHS was then added at a concentration of 1 mg/ml and allowed to react overnight at room temperature.

All prepared polymeric materials, including both PEGylated and bare nanoparticles and linear polymers, were then placed in dialysis bags (molecular weight cutoff=14,000 Da) and washed in a ddH₂O reservoir replenished twice daily for five days to remove any unreacted materials. The washed polymeric materials were next frozen overnight and lyophilized, and finally examined in dried powder form or resuspended in the appropriate buffer for further analysis.

Characterization

The kinetics of the polymerization and final conversion achieved by the various monomers used in the polymerization were determined using a differential scanning calorimeter (DSC 7, Perkin Elmer, Wellesley, Mass.). To obtain this data batches were prepared using the standard method described above; however, before initiating the polymerization, 60 μl of solution were transferred from the 3-neck round bottom flask to a large volume (60 μl) hermitically sealed DSC pan under an inert atmosphere. The pan was weighed before and after addition of the sample using a high accuracy analytical balance to determine the exact weight of the added DSC sample. The polymerization kinetics and conversion were then determined using the standard isothermal method previously described in the literature (25).

Briefly, the samples were heated from 25° C. to 60° C. at a rate of 100° C./min and held at 60° C. for 3 hours, during which time the thermal energy evolved by the polymerization was measured and recorded. After 3 hours the sample was then heated to 120° C. at a rate of 10° C./min and held for 15 minutes and then cooled at a rate of 10° C./min back to 25° C. This same ramp was then applied a second time, to establish a baseline heating profile, and the difference in thermal energy evolved between the two ramps was used as a measure of the unreacted monomer present in the system. Finally, the experimentally measured total energy evolved was also compared to theoretical total energy available based on the weight of monomers added to the DSC pan and heat of polymerization values found in the literature for the various monomers that were polymerized. Each monomer was run in triplicate to ensure the reproducibility of the calculated final conversion.

The morphology of the polymeric nanoparticles was examined using a field emission scanning electron microscope (FE-SEM, 1530, LEO, Oberkochen, Germany) operating at 10 kV. Purified samples were first frozen overnight and then lyophilized in a 4.5 liter manifold lyophilizer (Freezone, Labconco, Kansas City, Mich.). To prepare the samples for imaging, the polymeric nanoparticles in powder form were then mounted on an aluminum SEM stage using double-sided conductive carbon tape and coated with gold for 30 seconds using a sputter-coater (Model 3, Pelco, Redding, Calif.) in an argon atmosphere at a deposition rate of 10 nm/min.

The polyethylene glycol equivalent molecular weight and polydispersity index of the various linear polymer batches was determined using a HPLC (Waters, Milford, Mass.), equipped with Waters Ultra-Hydrogel 2000, 1000, 500, and 250 GPC columns maintained at 40° C. and a Waters 2414 refractive index (RI) detector, using a 4:1 by volume mixture of 0.1 molar NaNO₃ aqueous solution to acetonitrile mobile phase (26). Measurements were made by resuspending washed and dried linear polymer samples to a concentration of 15 mg/ml of mobile phase solution. The samples were then filtered with a 0.22 micron filter and injected into the GPC, along with poly(ethylene glycol) Mp 500-491,000 standards, using a 50 μl injection volume at a flow rate of 1 ml/min.

The hydrodynamic diameter of the particles in solution as a function of temperature and pH was determined using a dynamic light scattering (DLS, ZetaPlus, Brookhaven, Holtsville, N.Y.) instrument operating at a 90° scattering angle with a 635 nm 35 mW diode laser source. To obtain this data, washed and dried particles were resuspended in an aqueous buffer and their hydrodynamic diameter was measured every 2° C. from 25° C. to 55° C. For pH studies the hydrodynamic diameter was measured at 25° C. across a range of pHs from 2-9.

The surface charge of the PAAm/PAA IPN nanoparticles before and after PEGylation was examined using a laser doppler velocimeter (LDV, ZetaPlus, Brookhaven, Holtsville, N.Y.) instrument operating at a 90° scattering angle with a 635 nm 35 mW diode laser source and equipped with a dip-in Uzgiris type electrode system.

The infrared spectra of the polymeric nanoparticles was obtained in the wavenumber range of 400-4000 cm⁻¹ using a Fourier transform infrared spectrophotometer (FT-IR, Thermo Mattson Infinity, Thermo Electron Corp., Waltham, Mass.) in transmission mode equipped with a KBr beamsplitter and DTGS detector. To obtain this data, 1 mg of lyophilized polymeric material in powder form was thoroughly mixed with 150 mg of KBr and pressed into a pellet for analysis using a Carver laboratory press operating at 15,000 lbs compression force for minutes.

Results

Particle Size and Morphology

All of the monomers, the crosslinker, and the initiator that were used in the preparation of the various polymeric nanoparticles were soluble in water and were therefore present in small aqueous droplets and micelles spread throughout the cyclohexane continuous phase of the inverse emulsion. Upon heating the reaction mixture to the polymerization temperature of 60° C., initiation typically occurred within the smaller and more prevalent micelles; however, initiation can also occur in the larger monomer droplets leading to a higher degree of polydispersity in the final particle size distribution (27). As the reaction progressed, the smaller aqueous micelles exhausted their supply of monomer and crosslinker and diffusion away from the larger droplets to the growing micelles began to occur. As this happened the overall size of the particles began to increase and the solution became cloudy due to the increased size of the particles, which were now on the order of the wavelength of visible light. The reaction then continued to completion, typically within 2 hours of initiation of the polymerization.

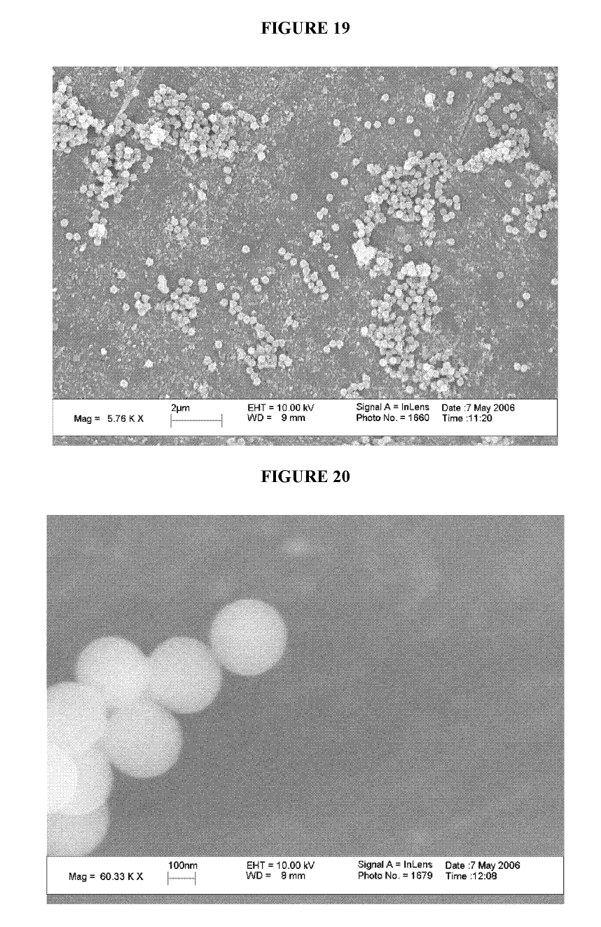

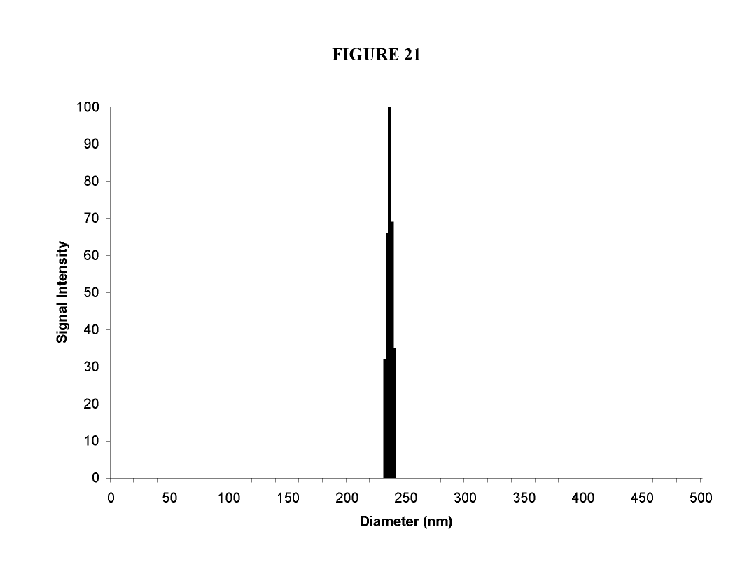

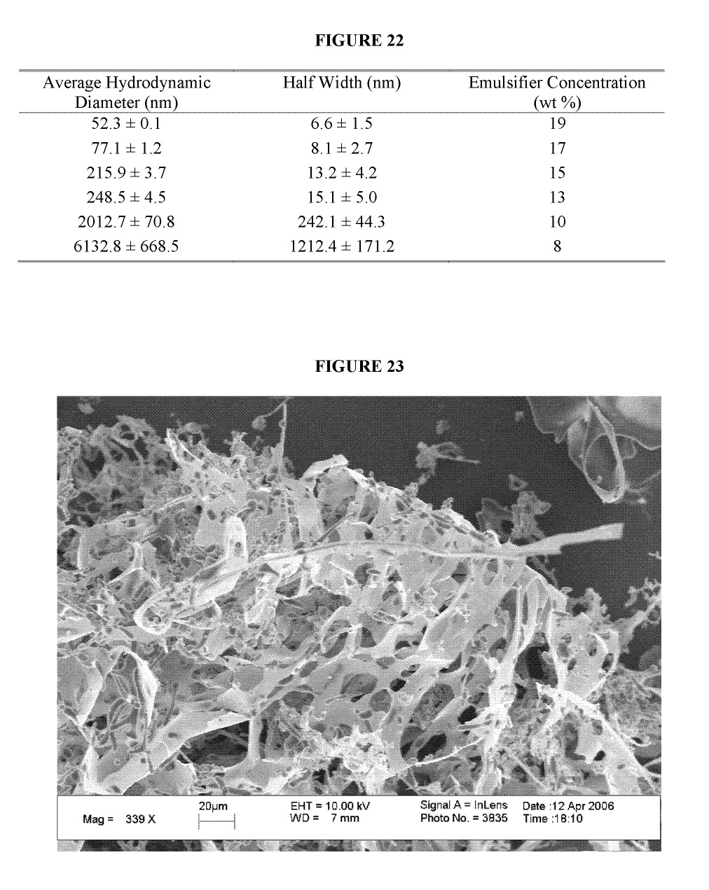

Scanning electron microscopy was utilized to determine the morphology of the particles which was spherical as shown in FIGS. 19 and 20. The particles also appeared to be monodisperse in size which was confirmed by examination of the particles size distribution using dynamic light scattering (FIG. 21). The final average particle size as function of emulsifier concentration was also examined and is listed in FIG. 22. The range of 19 wt % to 8 wt % emulsifier was explored due to the solubility limit of AOT in the cyclohexane phase at concentrations above 19 wt %. Also from experimental observation, it was apparent that emulsifier concentrations below 8 wt % no longer yielded discrete polymeric particles, but rather a discontinuous fragmented polymer film as shown in FIG. 23. From FIG. 22 it is apparent that as the amount of emulsifier in the system is decreased, the size and polydispersity of the particles prepared using this method tended to increase. Therefore, the particle size can be easily adjusted over a range of approximately 50-5000 nm diameter by controlling the percentage of emulsifier that is used in the emulsion system.

Polymerization Kinetics and Conversion

It has been shown (28) that the ratio of acrylamide repeat units to acrylic acid (or other analog) repeat units in the final IPN structure is critical to achieving enhanced thermally responsive properties. Specifically, the ability to obtain a sharp transition with temperature as well as a large volume change from the collapsed to swollen state is decreased as the final IPN structure moves further away from the ideal situation of a 1 to 1 molar ratio of repeat units (29, 30). Therefore, it is critical to know the exact conversion rate of the various monomers in this specific emulsion system and adjust the feed ratios accordingly to ensure that the final IPN structure will exhibit a one to one monomer ratio.

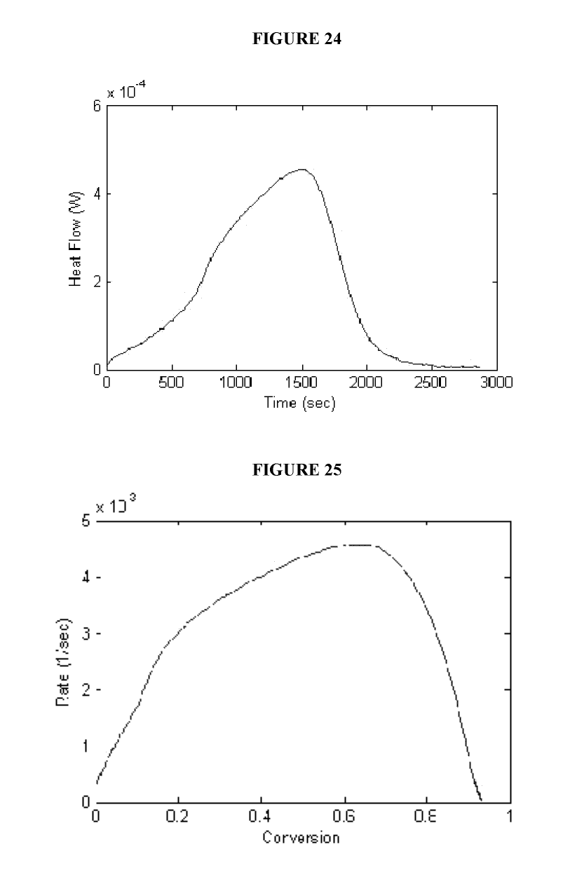

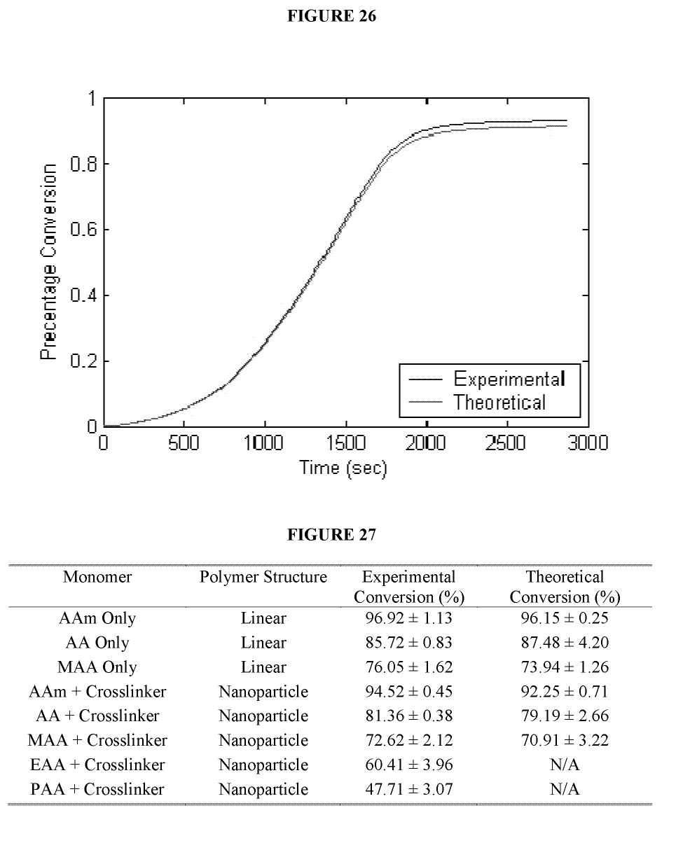

In order to ensure that this requirement was met, the various monomers were analyzed individually both with (1 mol % relative to the total amount of added monomer and crosslinker) and without added crosslinker using a differential scanning calorimeter to determine their reaction rate and final percentage conversion within the inverse emulsion polymerization system. FIGS. 24-26 are representative of the heat flow profiles, reaction rates, and percentage conversions, respectively, that were measured and calculated during the course of the polymerization. The results of this analysis are summarized in FIG. 27. Theoretical conversions were calculated using the mass of added monomer in a given experiment and the heats of polymerization values: 81.5 kJ/mol for AAm (25), 82.7 kJ/mol (of double bonds) for MBAAm (31), 77.5 kJ/mol for AA (32), and 66 kJ/mol for MAA (33). Values for EAA and PAA were not available in the literature. From FIG. 27, it is apparent that the first step of the IPN synthesis reaction involving the formation of the polyacrylamide portion of the network goes nearly to completion as evidenced by the high final percentage conversion of 94.52±0.45. Furthermore, it is also apparent that as the hydrophobicity of the monomer used in the polymerization structure is increased the overall percentage conversion decreased, which was most likely due to an increased partitioning of the monomer in the non-reactive continuous cyclohexane phase. These conversion results were then used to determine the feed ratios of the various monomers necessary to ensure as close to a one to one ratio of the monomer repeat units in the final IPN structure.

Molecular Weight Characterization

Inverse emulsion polymerization usually yields a relatively high molecular weight polymer. The polydispersity and molecular weight of polymers synthesized using this technique (27) are also almost completely independent of the concentration of initiator used. Therefore, to examine these effects, GPC analysis was performed on a series of linear polyacrylamide samples that were prepared using the inverse emulsion polymerization technique described previously with varying amounts of initiator.

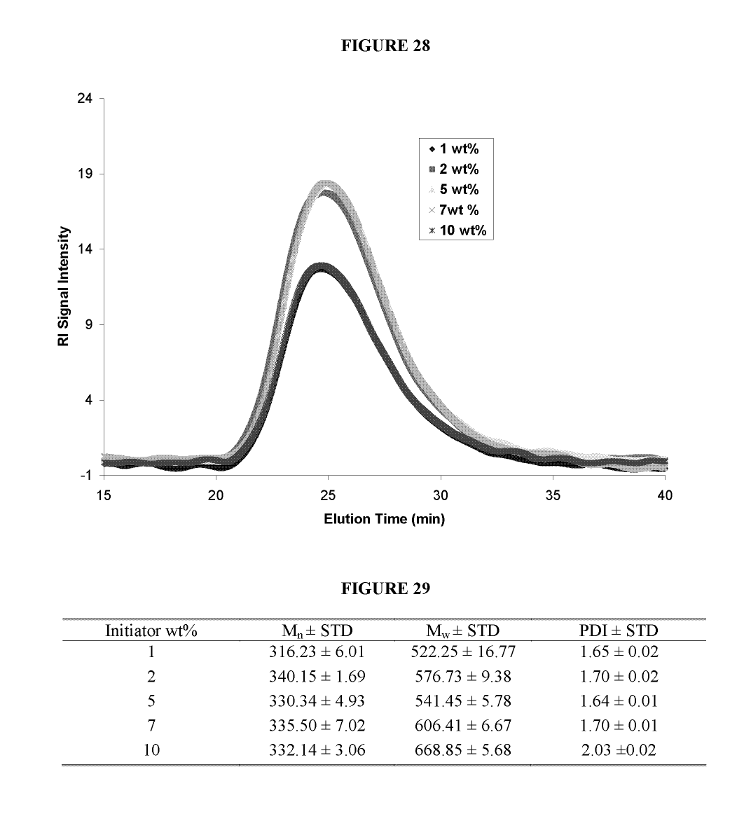

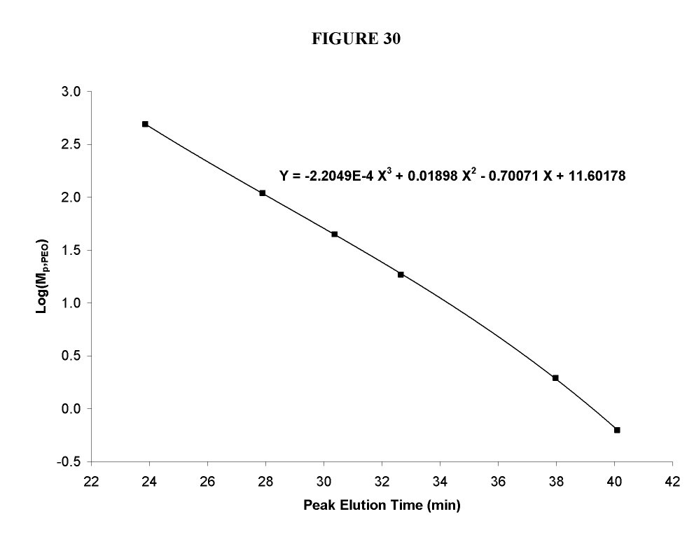

FIG. 28 shows the refractive index (RI) trace for a representative sample of linear polyacrylamide polymers that were synthesized with varying initiator concentrations. From this graph it is clear that even changing the initiator concentration by an order of magnitude from 1 wt % up to 10 wt %, relative to the total amount of added monomer and initiator, had little effect on the observed molecular weight and polydispersity of the linear polyacrylamide samples that were synthesized. Furthermore, FIG. 29 quantifies the actual changes in the PEO equivalent number average molecular weight (Mₙ), weight average molecular weight (M_{w}) and polydispersity index (PDI) of the various linear polyacrylamide samples that were synthesized. From this table there appears to be no discernable trend in molecular weight with initiator concentration; however, this data does show a slight increase in polydispersity with increasing initiator concentration. This trend is most likely due to the increased occurrence of initiation within the larger aqueous droplets due to the higher initiator concentration. FIG. 30 shows a typical PEO standard curve that was used to calculate the PEO equivalent molecular weight and PDI of the various linear polyacrylamide samples that were measured. New PEO standards were prepared and analyzed to calibrate each individual GPC analysis experiment.

Swelling Studies

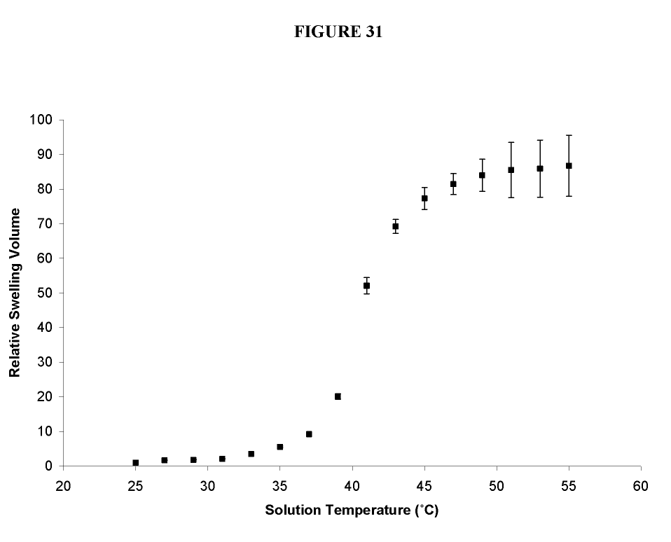

Dynamic light scattering was used to confirm the thermally-responsive upper critical solution temperature (UCST) behavior of the interpenetrating polymer network (IPN) nanoparticles. FIG. 31 illustrates the thermally responsive UCST behavior of a sample of polyacrylamide/poly(acrylic acid) IPN nanoparticles (0.1 mol % crosslinked) suspended in an aqueous pH 3 buffer solution. FIG. 31 shows the change in relative swelling volume (RSV), defined as the average volume of the swollen particles over the average volume of the collapsed particles, versus temperature. These results clearly illustrated the UCST like response of the IPN nanoparticles, as well as the very large final swollen volume that was achieved. Error bars in the graph represent one standard deviation, n=10, and generally tend to increase as the diameter of the particles increased. This trend was due to an overall decrease in the optical density and scattering efficiency of hydrogel particles in the swollen state, which leaded to higher variability in the measured hydrodynamic diameter.

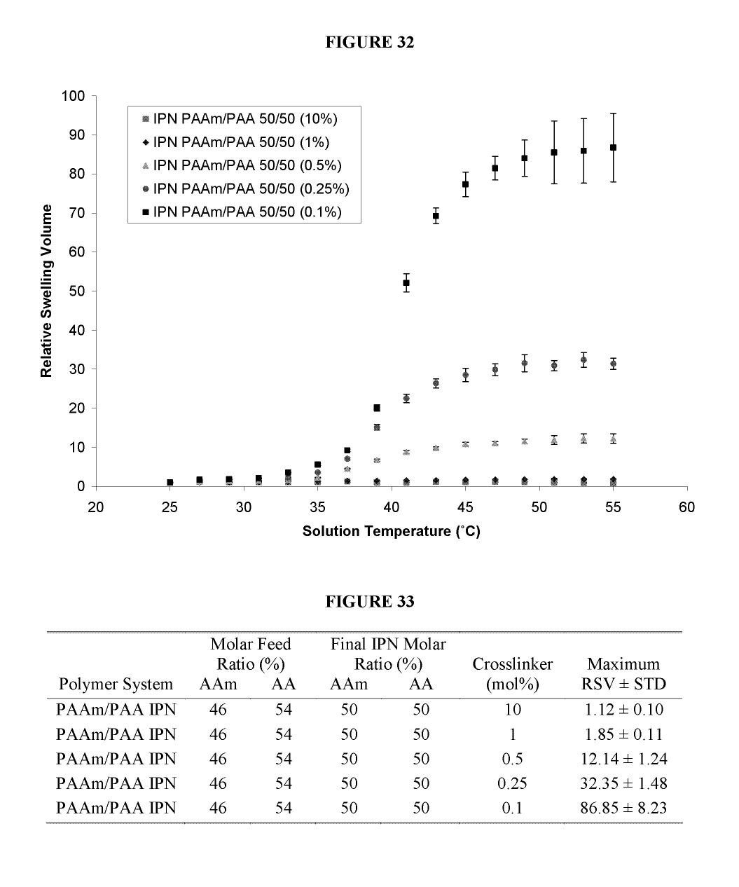

The effect of crosslinker concentration on the thermally-responsive swelling properties of a series IPN nanoparticle samples, suspended in an aqueous pH 3 buffer solution, is clearly illustrated in FIG. 32. The monomer ratios and molar percentages of crosslinker used to prepare these IPN nanoparticles, as well as the maximum RSV achieved, are listed in FIG. 33. All the IPN particles described in this table were synthesized with an initiator concentration of 7 wt % with respect to the total weight of the monomer and crosslinker. The molar feed ratios listed in Table 4.4 were based on the ratio of the combined total of AAm to AA added to the emulsion system in both the first and second steps of the IPN nanoparticle synthesis procedure. The monomer conversion rates, determined previously using DSC, were then utilized to calculate the theoretical ratio of AAm to AA repeat units present in the final IPN nanoparticle structure. The molar percentages of crosslinker were based on the total moles of both monomer and crosslinker used to prepare an individual polymer network and were always maintained at the same level in both networks for any given IPN system. For instance, a PAAm/PAA IPN crosslinked at 0.1 mol %, would be comprised of a polyacrylamide network that was prepared with 0.1 mol % crosslinker as well as a poly(acrylic acid) network that was also prepared with 0.1 mol % crosslinker. From this analysis it is clear that PAAm/PAA IPN nanoparticles prepared with a 50/50 AAm to AA ratio of repeat units in the final IPN polymer structure were able to achieve UCST-like swelling behavior. It is also evident from this graph that increased molar percentage of crosslinker lead to a decrease in the final volume swelling ratio of the IPN particles.

The effect of pH on the thermally-responsive properties of these systems is critical because as the pH is increased above the pKa of the poly(acrylic acid) or poly(acrylic acid) homolog portion of the IPN the carboxyl groups of that polymer become deprotonated. Once this occurs, a strong charge repulsion force will drive the immediate swelling of the polymer network. Furthermore, the deprotonation of the carboxylic acid group limits the hydrogen bonding capabilities of the IPN system and, in effect, destroys its thermally-responsive behavior.

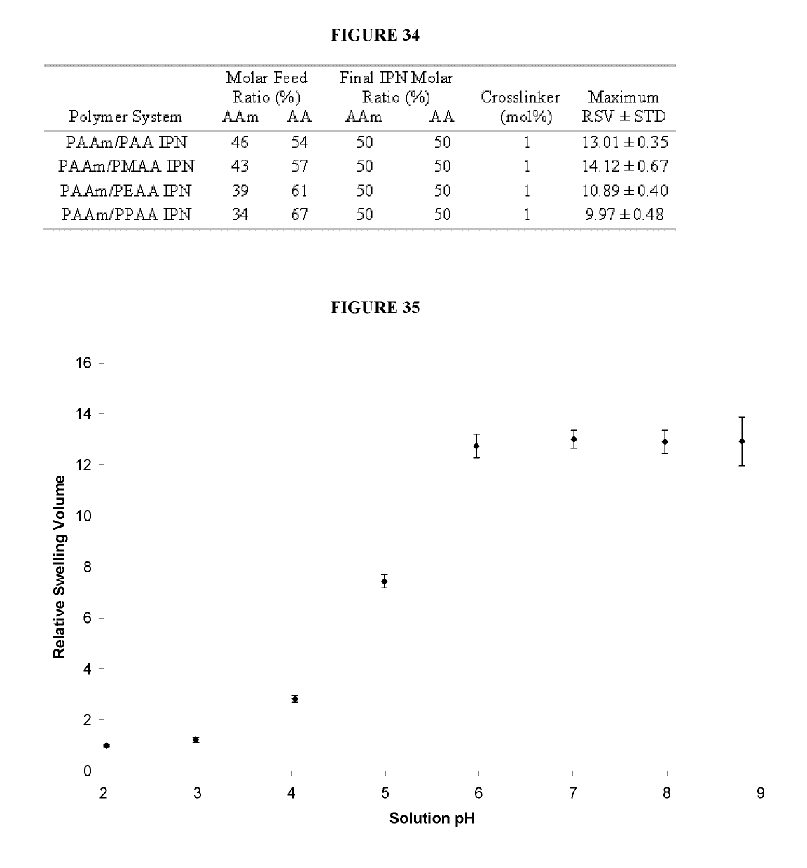

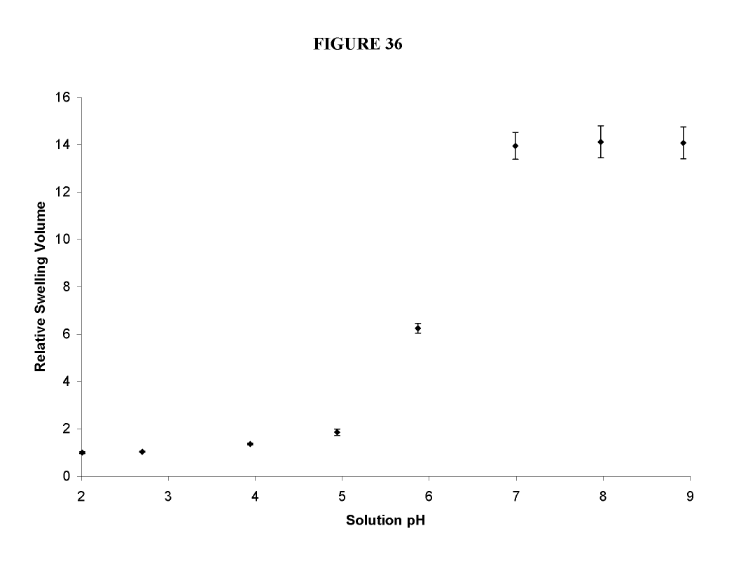

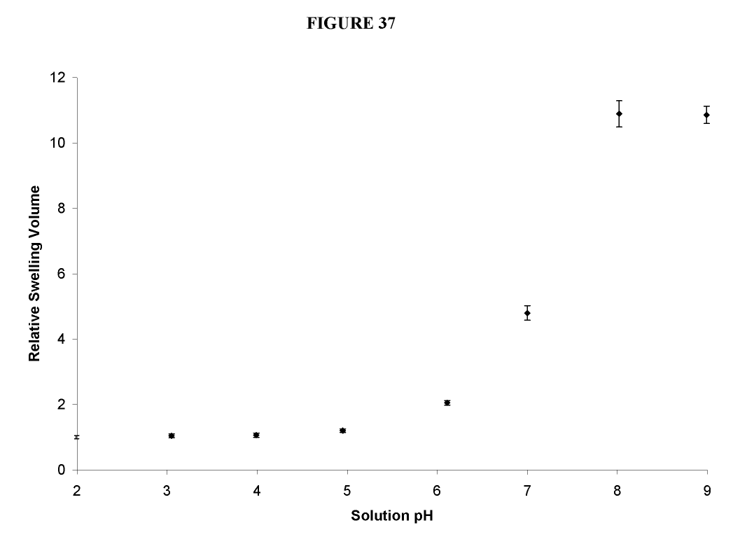

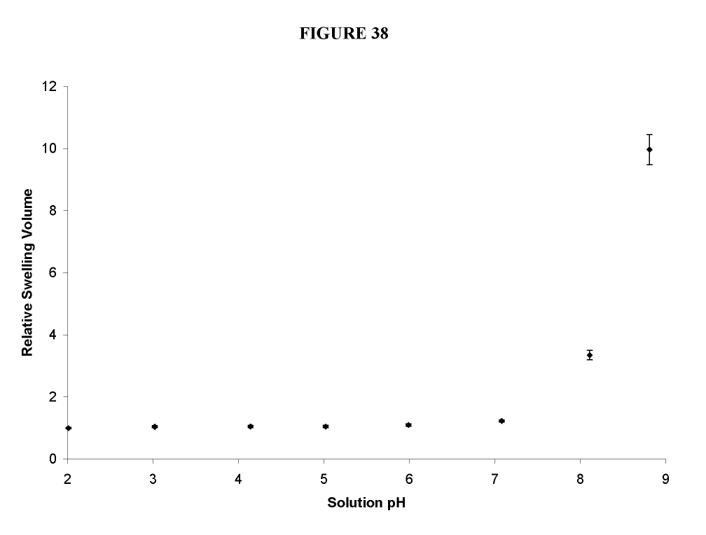

Therefore, the effect of pH on the swelling properties of IPN nanoparticles comprised of polyacrylamide and poly(acrylic acid) and its homologs was investigated using DLS. FIG. 34 lists the various IPN nanoparticles that were investigated, the maximum RSV that was obtained for each system, and the monomer ratios and molar percentage of crosslinker used to prepare the various IPN nanoparticles. All particles used in this study were also prepared with 7 wt % initiator as described previously. FIG. 35 shows the trend in the RSV with pH that was observed for the PAAm/PAA IPN nanoparticles. From this graph it is clear that the particles began to become deprotonated and swell in the pH range of 4-5 and were fully swollen, and hence fully deprotonated, at a pH of 6. This corresponds well with what is expected based on a pKa of 4.8 for PAA as reported in the literature (34). FIG. 36 shows the trend in RSV with pH that was observed for PAAm/PMAA IPN nanoparticles. From this graph it is clear that the pH of initial deprotonation was shifted to a value in the pH range of 5-6. This also corresponds with the literature value of 6.15 for the pKa of PMAA (35). The pKa of PEAA is listed as 7.2 in the literature (35) and FIG. 37 confirms that the initial deprotonation and swelling of PAAm/PEAA IPN nanoparticles occurred in the pH range of 6-7 as expected. PAAm/PPAA IPN nanoparticles were also investigated and showed an even further initial deprotonation shift in the pH range of 7-8 (FIG. 38). Although no exact PPAA pKa value was available in the literature; articles have shown a significant increase in the pH required for the deprotonation of PPAA as compared to PEAA (36).

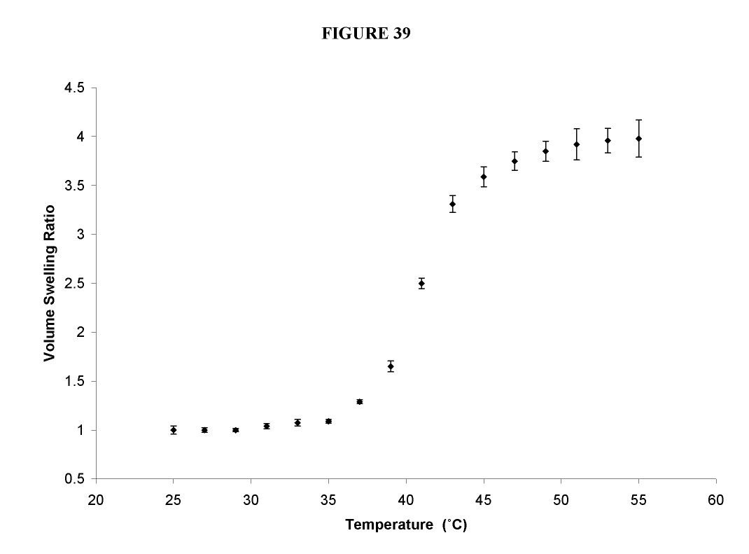

In order to be effective in vivo, IPN nanoparticles will need to be able to swell in response to heating in physiological conditions (i.e. at pH 7.4 and 150 mM ionic strength). To test these conditions a sample of the same PAAm/PPAA IPN nanoparticles tested in the previous pH study were resuspended in a pH 7.4 Phosphate Buffered Saline (PBS) solution at an ionic strength of 150 mM. The system was then heated as before from 25° C.-55° C. with the hydrodynamic radius measured every 2° C. The results of this analysis are shown in FIG. 39. From this graph it is clear that the particles were able to exhibit a UCST-like swelling transition even in the much higher pH and ionic conditions of the PBS buffer system with a maximum volume swelling ratio of 3.98±0.19.

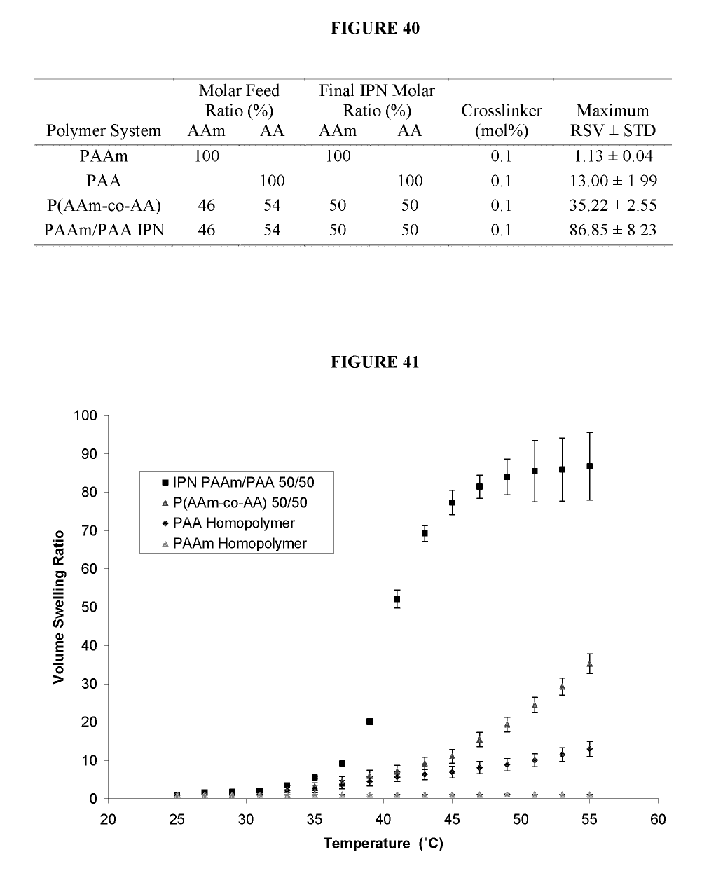

The effect of polymeric structure on the swelling properties of various polyacrylamide and poly(acrylic acid) nanoparticles, suspended in a pH=3 aqueous buffer, was also investigated using DLS. The monomer ratios and molar percentages of crosslinker used to prepare these polymer nanoparticles, as well as the maximum RSV achieved, are listed in FIG. 40. In this study homopolymer nanoparticles of both polyacrylamide and poly(acrylic acid) were compared to a random copolymer of polyacrylamide-co-poly(acrylic acid) as well as polyacrylamide/poly(acrylic acid) IPN. Furthermore, in the case of the random copolymer and IPN, both were prepared in such a way to ensure that the ratio of AAm to AA repeat units in the final polymer structures was one to one. The results of the study are shown graphically in FIG. 41. From this graph it is clear that the IPN polymer structure yielded a much large maximum RSV as well as a sharper UCST-like swelling transition. The random copolymer, as well as the poly(acrylic acid) homopolymer, exhibited some swelling with temperature and the homopolymer polyacrylamide particle exhibited almost no swelling with increased temperature.

Zeta Potential Analysis

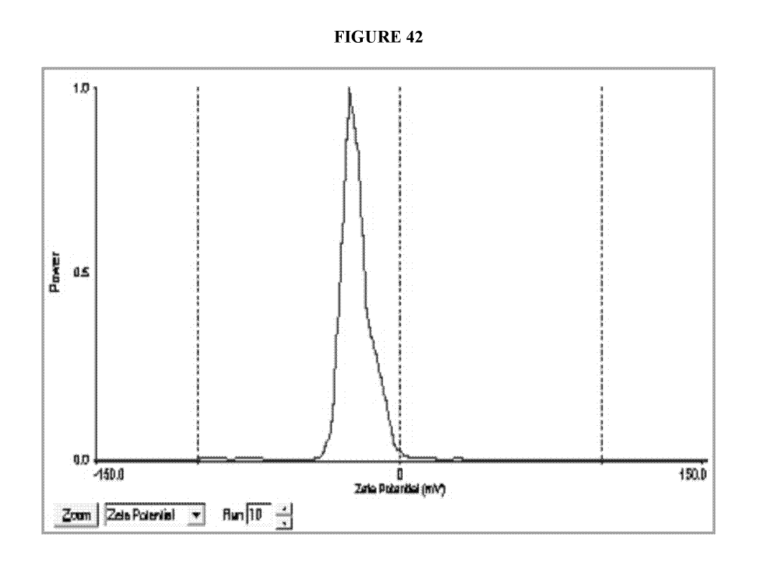

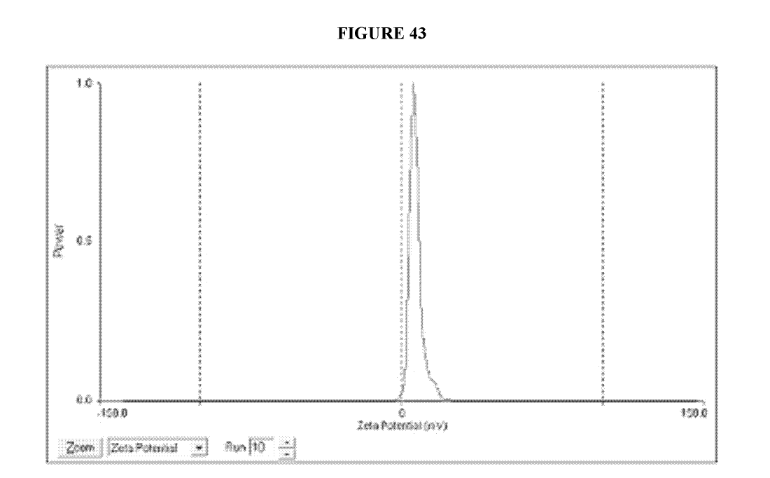

Research has shown that bare polymeric nanoparticles when injected in vivo are quickly recognized and removed by the body's natural defensive systems (22, 23, 37). However, the successful PEGylation of polymeric nanoparticles can dramatically increase their biocompatibility and blood circulation half life (18, 21). Furthermore, the covalent attachment of PEG chains on the surface of polymeric nanoparticles creates a steric repulsive layer around the particles, thereby increasing their stability in solution. Therefore, to increase the stability and biocompatibility of the polymeric nanoparticles, large molecular weight poly(ethylene glycol) chains were covalently bound to their surface using a N-hydroxysuccinimide functionalized PEG chain. Zeta potential was used to confirm the successful PEGylation of the IPN nanoparticles. FIG. 42 is a representative Zeta potential analysis of an as-prepared batch of PAAm/PAA IPN nanoparticles showing a negative surface charge, due to the ionization of carboxylic acid groups present in the poly(acrylic acid) portion of the IPN, of −19.16±3.89 mV (n=10). FIG. 43 is a representative Zeta potential analysis of the PEGylated PAAm/PAA IPN nanoparticles showing an approximately neutral to slightly positive surface charge, due to charge masking by the neutral PEG surface layer, of 2.77±1.08 mV (n=10).

FT-IR Spectroscopy of IPN Polymer Nanoparticles

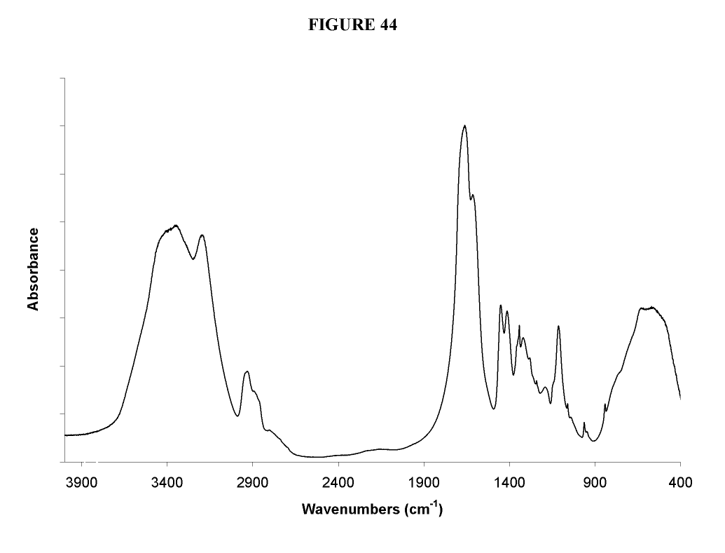

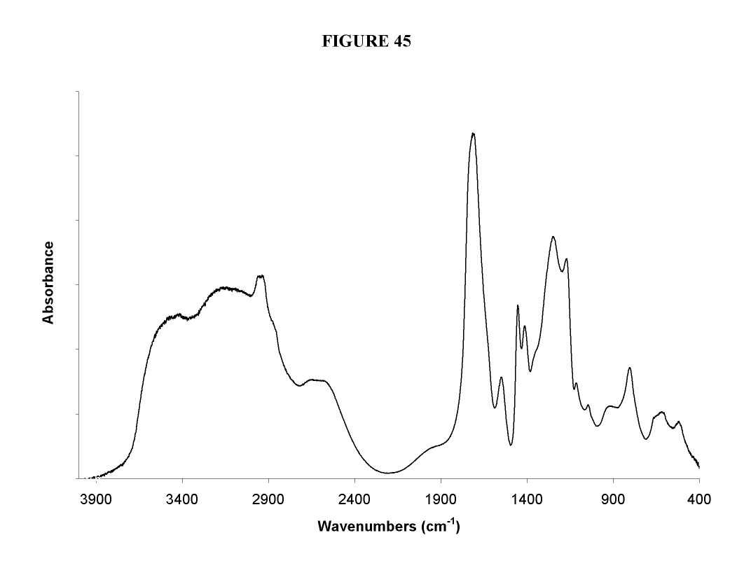

The molecular structure of the IPN polymeric nanoparticles was investigated using FT-IR. FIG. 44-45 show the FT-IR spectrum of crosslinked homopolymer nanoparticles of PAAm and PAA respectively. In FIG. 44 the characteristic absorption bands of polyacrylamide were present at 3360 and 3210 cm⁻¹ corresponding to the asymmetric and symmetric NH₂ stretching vibrations, 2945 cm⁻¹ corresponding to the stretching of CH₂ group, and 1665 cm⁻¹ corresponding to the stretching of the C═O group. There were also weaker bands at 1455 and 1420 cm⁻¹ associated with scissor and bending vibrations of CH₂ and CH—CO groups, respectively. Finally, the weak bands in the range of 1050 to 1350 cm⁻¹ and 750 to 850 cm⁻¹ corresponded to the stretching vibrations of C—N and the out-of-plane bend of NH₂, respectively (38).

In FIG. 45 the characteristic absorption bands of poly(acrylic acid) were present at 1720 cm⁻¹ corresponding to the stretching of the C═O group, the broad band between 1180 and 1260 cm⁻¹ corresponding to the stretching of C═O coupled with the bending of O—H groups, and the broad band from 3100 to 3500 corresponding to the stretching of the O—H group with a peak at 3200 cm⁻¹, and the free O—H group with a peak at approximately 3450 cm⁻¹. There were again weaker bands at 1456 and 1415 cm⁻¹ associated with scissor and bending vibrations of CH₂ and CH—CO groups, respectively. Finally, the weak bands at 2960 and 2635 cm⁻¹ corresponded to the stretching vibrations of CH₂ and O—H bonded groups, respectively (38, 39).

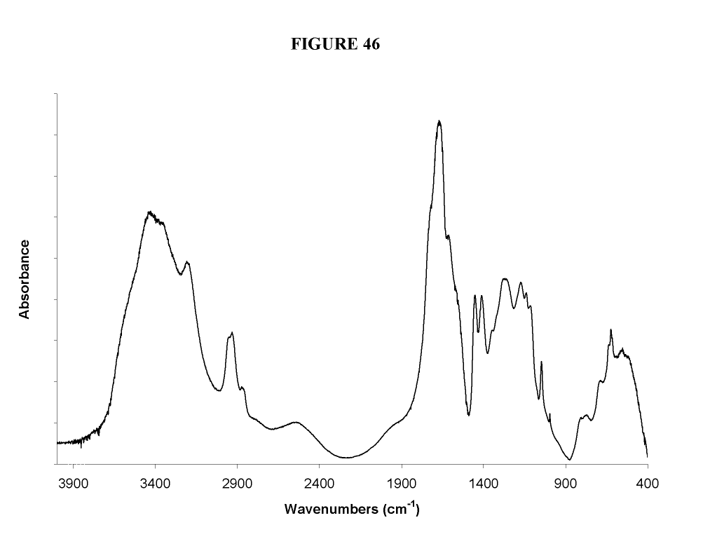

In FIG. 46 a mixture of absorption bands from both the PAAm and PAA portions of PAAm/PAA IPN nanoparticles was evident. A broad and shifted C═O band at 1680 cm⁻¹ was due to the combined stretching vibrations of the C═O groups of both PAAm and PAA as well as the effects of hydrogen bonding present in the IPN structure. Absorption bands at 1455 and 1416 cm⁻¹ were again associated with scissor and bending vibrations of CH₂ and CH—CO groups in both PAAm and PAA. Furthermore, the absorption band at 2950 cm⁻¹ corresponded to the combined stretching of CH₂ groups in both PAAm (2945 cm⁻¹) and PAA (2960 cm⁻¹). Finally, the broad absorption bands in the 3100 to 3500 cm⁻¹ regions were due to the overlapping absorption bands of O—H and NH₂ stretching vibrations, while the absorption bands in the 1150 to 1300 cm⁻¹ region were due to the overlap of the stretching C—N, and C═O coupled with the bending of O—H groups (38, 40). Therefore, the results of this analysis further confirmed the presence of both poly(acrylic acid) and polyacrylamide in the final IPN structure, which was expected based on previous DLS swelling studies and DSC conversion studies of these materials.

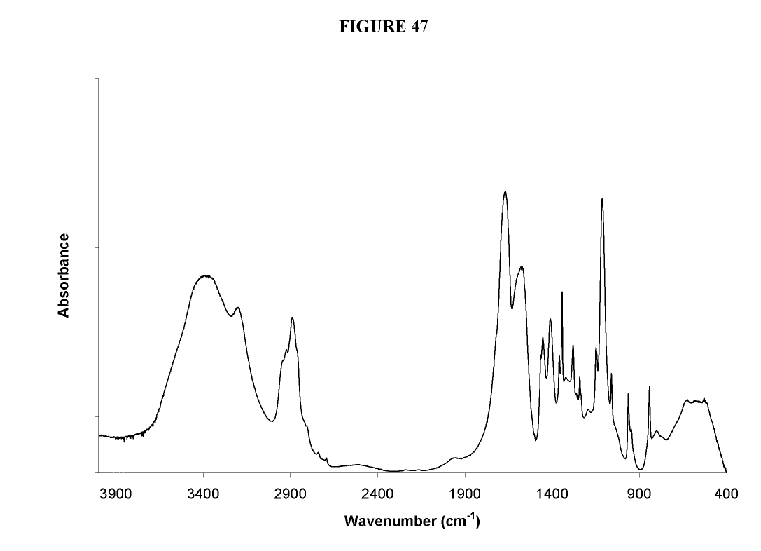

The FT-IR absorption spectrum of the IPN nanoparticles after PEGylation (FIG. 47) showed similar absorption bands to the IPN nanoparticles before PEGylation including the broad bands from 3100 to 3500 cm⁻¹ and the bands at 1456 and 1415 cm⁻¹. However, two new strong bands were present at 1588 cm⁻¹, corresponding to the shift in the C═O band of polyacrylamide portion of the IPN, due to the selective binding of high molecular weight PEG to the primary amine group in this polymer, and 1115 cm⁻¹ corresponding to the characteristic asymmetric stretching vibration of the C—O—C group of the grafted PEG (38). Therefore, the results of this analysis further confirmed the presence of PEG in the final PEGylated IPN structure, which was expected based on previous Zeta potential studies of these materials.

The above-example demonstrates that thermally-responsive polymeric nanoparticles comprised of polyacrylamide and poly(acrylic acid) and its various (methyl-, ethyl-, and propyl-) analogs can be successfully synthesized using an inverse microemulsion polymerization technique. SEM and DLS confirmed the spherical morphology and monodisperse size distribution of polymeric nanoparticles prepared using this method.

DSC studies were conducted to determine the percentage conversion obtained for each monomer polymerization using this method. From this analysis it was evident that increasingly hydrophobic monomers achieved lower rates of conversion due to a higher partitioning in the non-reacting cyclohexane continuous phase. Furthermore, the results of these studies were used to formulate IPN and random copolymers in such a way that the final polymer structures would contain a one to one ratio of acrylamide to acrylic acid (or other acrylic acid analogs) repeat units.

DLS was used to confirm the UCST-like behavior of the IPN nanoparticles and the increased maximum relative swelling volume obtained by this polymer structure compared to random copolymer and homopolymer structures of similar size and composition. The effect of crosslinker on the UCST and maximum relative swelling volume of these systems was also elucidated. The effect of pH on various poly(acrylic acid) and poly(acrylic acid) homolog based IPNs was investigated and illustrated the dependence of the thermally-responsive swelling properties of these systems on the pKa of the polymer system utilized. Furthermore, it was also illustrated that a PAAm/PPAA IPN nanoparticle can obtain UCST-like swelling behavior in physiologically relevant (i.e. pH 7.4 and 150 mM ionic strength) conditions.

Finally, FT-IR analysis was used to further confirm the presence of PAAm and PAA groups in the final PAAm/PAA IPN nanoparticles, and FT-IR and Zeta potential analysis were both used to confirm the successful PEGylation of PAAm/PAA IPN nanoparticles using a heterofunctional acryl-poly(ethylene glycol)-NHS (MW=5,000).

Metal-Polymer Nanocomposite Synthesis and Characterization

Materials

Acrylic acid (AA, inhibited with 200 ppm hydroquinone monomethyl ether), N,N′-methylenebisacrylamide (MBAAm), polyethylene glycol lauryl ether (Brij 30), cyclohexane, sodium bis(2-ethylhexyl) sulfosuccinate (AOT), and sodium citrate tribasic dehydrate were obtained from Sigma Aldrich (Milwaukee, Wis.), acrylamide (AAm) and ammonium persulfate (APS) were obtained from Fisher Scientific (Hampton, N.H.), chloroauric acid was obtained from Acros Organics (Geel, Belgium), and acryl-poly(ethylene glycol)-N-hydroxysuccinimide (MW=5,000) was obtained from Nektar Therapeutics (San Carlos, Calif.). All were used as received for the preparation of gold metal-polymer nanocomposite particles.

Synthesis

Solid gold nanoparticles (˜50 nm diameter) were prepared via the common technique of citrate reduction, which has been previously described in detail (13). Briefly, a 50 ml solution of 0.25 mM chloroauric acid was prepared in a round bottom flask and dark aged overnight. The chloroauric acid solution was then brought to a boil under reflux and 0.5 ml of 40 mM sodium citrate was injected into the round bottom flask under vigorous stirring. This solution was allowed to react for 1 hour at room temperature and then capped with the addition of enough mPEG-SH to achieve a final concentration of 1 μM. These PEG functionalized gold nanoparticles were then pelleted out and washed three times using centrifugation (3000 rcf for 30 min) to remove any excess chloroauric acid or mPEG-SH.

Polymer-gold nanocomposites were then formed using a two-step, sequential IPN synthesis method. First, gold nanoparticles were encapsulated inside of polyacrylamide nanoparticles via an in situ inverse emulsion polymerization method with the gold nanoparticles located in the aqueous monomer droplet phase. This inverse emulsion solution consisted of an 81% cyclohexane continuous phase, with a 13% surfactant phase (AOT and Brij 30 in a 2:1 ratio), and a 6% aqueous phase. In a typical experiment, 1 ml of previously prepared PEGylated gold nanoparticles suspended in aqueous solution at a concentration of ˜1×10¹² particles/ml were added directly to a 3-neck round bottom flask containing the entire cyclohexane continuous phase and equipped with a condenser, nitrogen purge line, and overhead mechanical stirrer. To this, the entire emulsifier phase was added and dissolved under vigorous stirring. For the first stage of the sequential IPN polymerization, only the acrylamide monomer along with crosslinker, imitator, and deionized distilled water (ddH₂O) was added. This mixture was then purged with nitrogen gas for 30 minutes to remove oxygen and homogenized (Ultra-Turrax T25, IKA, Willmington, N.C.) at 24,000 rpm for 5 minutes. After homogenization the polymerization was then initiated thermally by immersion of the reaction vessel in a 60° C. bath and allowed to react to completion (typically 2 hours). Upon completion of the first stage of the IPN synthesis method, the second stage was then started by the addition of the other half of the aqueous phase, consisting of additional crosslinker, initiator, and acrylic acid, to the same 3-neck round bottom flask as before. The vessel was again purged with nitrogen gas, homogenized, and allowed to react at 60° C. for two hours, thus resulting in the formation of the complete gold core-PAAm/PAA IPN shell metal-polymer nanocomposite particles.

These metal-polymer nanocomposite particles were then collected and purified by removal of the cyclohexane phase with elevated temperature and reduced pressure (40° C./50 mmHg) in a rotary evaporator (RE-121, Buchi, Flawil, Switzerland). This was followed by precipitation of the particles out of the emulsifier phase with the addition of excess ethanol and subsequent pelting and washing (three times) by centrifugation (Centra CL3R, Thermo IEC, Waltham, Mass.) at 3200 rcf for 60 minutes. The purified metal-polymer nanocomposite pellet was then resuspended in deionized water for PEGylation.

The resuspended metal-polymer nanocomposite particles were then PEGylated using standard N-hydroxysuccinimide (NHS) chemistry (14). In a typical experiment, the pH of an aqueous suspension of metal-polymer nanocomposite particles, at a concentration of approximately 1 mg of composite material per ml of ddH₂O, was raised to the range of 7.5-8.5. A heterofunctional acryl-poly(ethylene glycol)-NHS (MW=5,000) was then added at a concentration of 1 mg/ml and allowed to react overnight at room temperature.

After PEGylation overnight, the PEGylated metal-polymer nanocomposite particles were then placed in dialysis bags (molecular weight cutoff=14,000 Da) and washed in a ddH₂O reservoir replenished twice daily for five days to remove any unreacted materials. The final washed PEGylated metal-polymer nanocomposite particles were then refrozen and lyophilized, and examined in dried powder form or resuspended in the appropriate buffer for further analysis.

Characterization

The morphology of the metal-polymer nanocomposite particles was examined using a LEO 1530 field emission scanning electron microscope (FE-SEM, Oberkochen, Germany) operating at 10 kV. Aqueous samples were first frozen overnight and then lyophilized in a 4.5 liter manifold lyophilizer (Freezone, Labconco, Kansas City, Mich.). The nanocomposite particles in powder form were then mounted on an aluminum SEM stage using double-sided conductive carbon tape and coated with gold for 30 seconds using a sputter-coater (Model 3, Pelco, Redding, Calif.) in an argon atmosphere at a deposition rate of 10 nm/min.

The internal structure and atomic composition of the metal-polymer nanocomposite particles was examined using a JOEL 2010F high resolution transmission electron microscope (HR-TEM, Tokyo, Japan) operating at 200 kV, with an attached Oxford INCA (Concord, Mass.) energy dispersive spectroscopy (EDS) detector with a 136 eV resolution. Samples were prepared for HR-TEM and EDS examination by drop drying of aqueous particle suspensions directly onto carbon coated 300 mesh copper TEM grids. Typically 10 μl of sample was allowed to dry overnight before examination in the HR-TEM.

The surface charge of the metal-polymer nanocomposite particles before and after PEGylation was examined using a laser doppler velocimeter (LDV, ZetaPlus, Brookhaven, Holtsville, N.Y.) instrument operating at a 90° scattering angle with a 635 nm 35 mW diode laser source and equipped with a dip-in Uzgiris type electrode system.

The relative size distribution of metal-polymer nanocomposite particles was determined using a dynamic light scattering (DLS, ZetaPlus, Brookhaven, Holtsville, N.Y.) instrument operating at a 90° scattering angle with a 635 nm 35 mW diode laser source. This same instrument was also used to examine the change in hydrodynamic diameter of the nanocomposite particles as a function of both time and external laser excitation. To obtain this data metal-polymer nanocomposite particles were suspended in an acidic (pH=3) buffer solution and placed inside a quartz cuvette that was then loaded into the DLS instrument. A 5 ns pulsed Q-switched Neodymium-doped Yittrium Aluminum Garnet (Nd-YAG) laser (Polaris II-20, New Wave, Fremont, Calif.) operating at the second harmonic of 532 nm was then used to excite the sample at 20 Hz. An optical diffuser was utilized to uniformly irradiate the cuvette from above, providing a fluence of 20 mJ/cm². The hydrodynamic diameter of the metal-polymer nanocomposites was then measured for 30 minutes with a 10 minute excitation laser off-period at the start followed by a 10 minute excitation laser on-period, and finally another 10 minute excitation laser off-period at the end.

The metal-polymer nanocomposite particles were also photoacoustically imaged in solution using the same Nd-YAG laser as in the swelling studies operating at 20 Hz and at the second harmonic of 532 nm to excite the sample and a 128 element linear array ultrasound transducer (Sonix, Ultrasonix Medical Corp, Burnaby, Canada) with a 5 MHz center frequency to detect the photoacoustic sound waves produced by the excited metal-polymer nanocomposite particles. Photoacoustic signal was collected by the ultrasound transducer over 75 μs and converted into a digital image based on the signal's intensity and speed of propagation.

Results

Nanocomposite Particle Size and Morphology

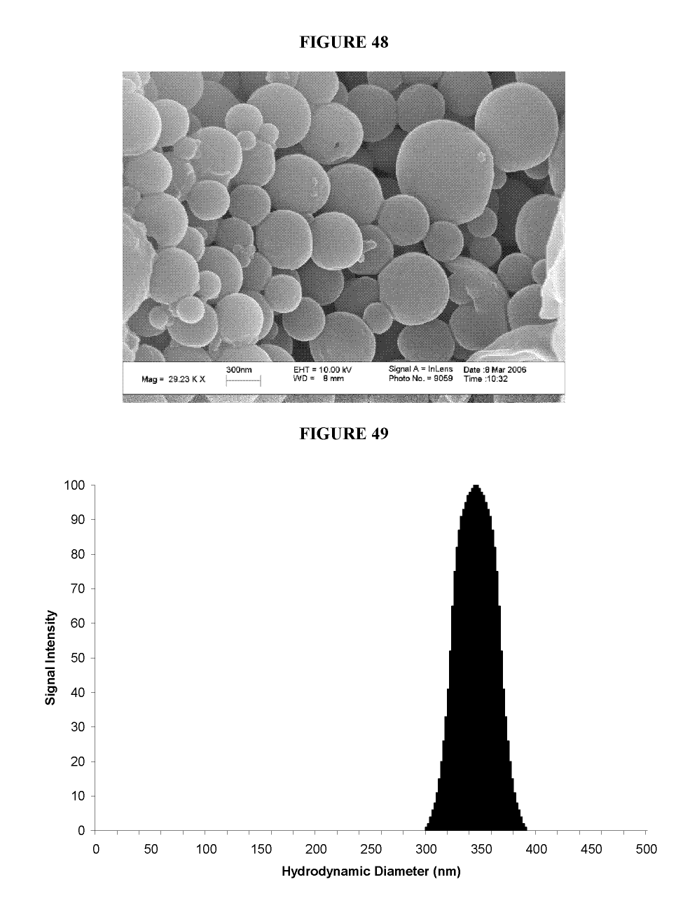

The size and morphology of the metal-polymer nanocomposite particles were examined using DLS and SEM imaging. FIG. 48 is an SEM micrograph of dried and gold-sputter coated nanocomposite particles which clearly illustrates their spherical morphology. It is also apparent from this image that the nanocomposite particles are somewhat polydisperse in size due to the random nature of the gold encapsulation process, whereby some polymer particles contain one or more encapsulated gold particles while others contain none. This polydispersity is apparent as well in the DLS analysis of the distribution of hydrodynamic diameters present in the metal-polymer nanocomposite particles shown graphically in FIG. 49.

Nanocomposite Composition

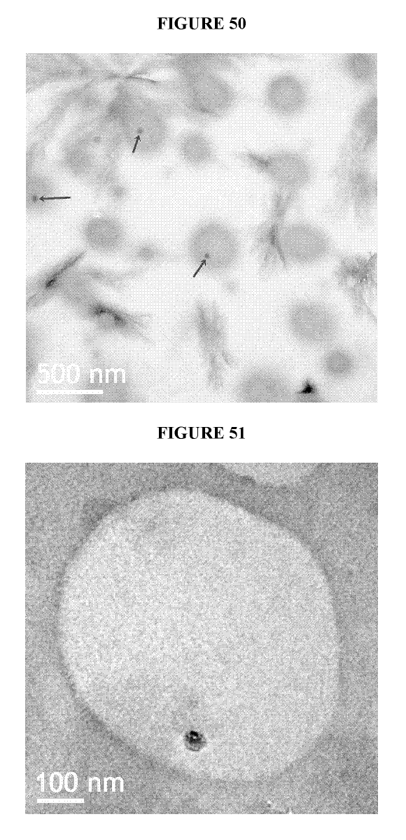

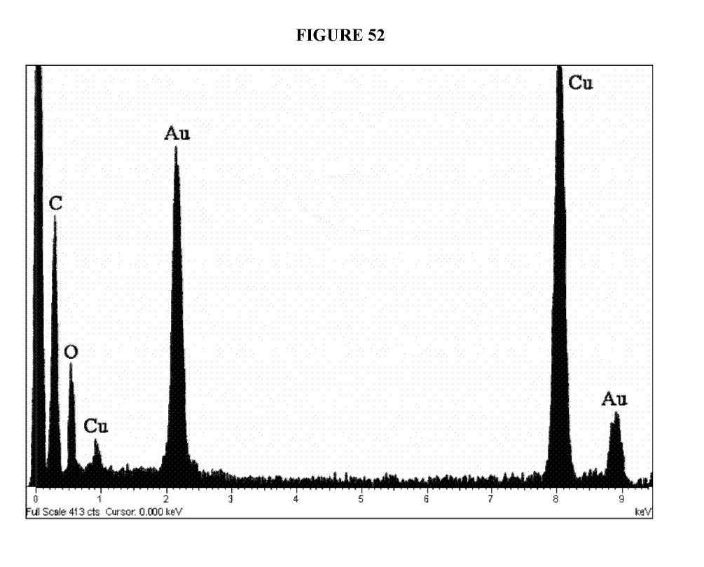

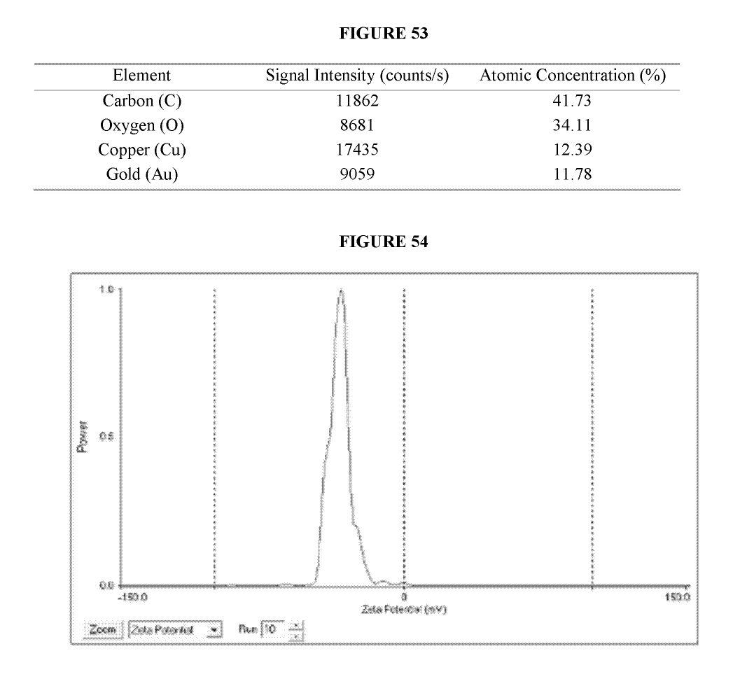

The internal structure and atomic composition of these particles was examined using TEM imaging analysis and single particle EDS spectroscopic analysis. FIGS. 50-51 clearly illustrate the presence of small solid gold nanoparticles encapsulated inside of larger polymeric nanoparticles. Specifically, in FIG. 50 red arrows indicate the presence of smaller gold nanoparticles which appear darker because they are more electron dense and hence block more of the electron beam during imaging than the larger light grey spheres which are the polymeric particles. FIG. 50 is a higher magnification TEM micrograph of the metal-polymer nanocomposite particle in the center of FIG. 6.4. FIG. 52 is a representative EDS analysis of the same metal-polymer nanocomposite particle in the center of FIG. 50. A small part of the carbon signal and all of the copper signal in the spectrograph are due to the carbon coated TEM grid on which the sample is mounted, the remainder of the carbon signal as well as all of the oxygen signal are due to the polymer portion of the nanocomposite particle, and the gold peak is entirely due to the gold particle encapsulated inside of the nanocomposite. The atomic composition of this metal-polymer nanocomposite particle as determined by single particle EDS spectroscopy is listed in FIG. 53.

Zeta Potential Analysis

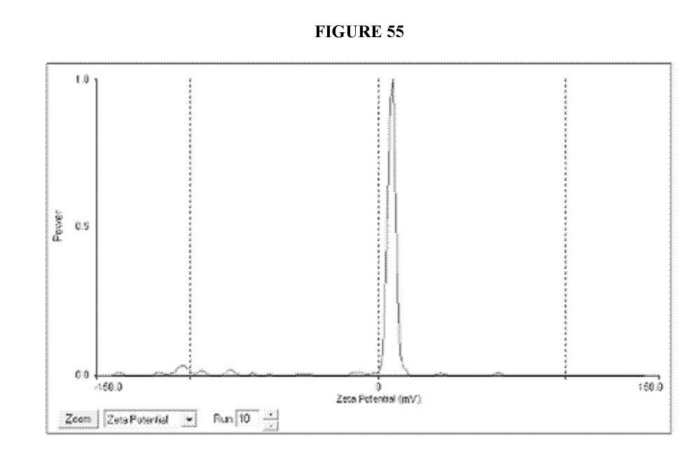

Research has shown that bare polymeric nanoparticles when injected in vivo are quickly recognized and removed by the body's natural defensive systems (15-17). However, the successful PEGylation of polymeric nanoparticles can dramatically increase their biocompatibility and blood circulation half life (18, 19). Furthermore, the covalent attachment of PEG chains on the surface of the metal-polymer nanocomposite materials creates a steric repulsive layer around the particles, thereby increasing their stability in solution. Therefore, to increase the stability and biocompatibility of metal-polymer nanocomposite particles, large molecular weight poly(ethylene glycol) chains were covalently bound to the surface of the gold nanoparticles using an N-hydroxysuccinimide functionalized PEG chain. FIG. 54 is a representative Zeta potential analysis of an as prepared batch of metal-polymer nanocomposite particles showing a negative surface charge, due to the ionization of carboxylic acid groups present in the poly(acrylic acid) portion of the IPN, of −23.54±4.15 mV (n=10). FIG. 55 is a representative Zeta potential analysis of PEG surface grafted metal-polymer nanocomposite particles showing an approximately neutral surface charge, due to charge masking by the neutral PEG surface layer, of 3.01±1.32 mV (n=10).

Laser Induced Swelling

The effect of excitation by a laser light source on the hydrodynamic diameter of the metal-polymer nanocomposite particles was examined using a dynamic light scattering instrument coupled with an external 532 nm Nd-YAG laser. The DLS instrument utilizes a 635 nm laser light source to measure the hydrodynamic diameter of particle suspensions. The instrument also has built-in optical filters to ensure that only wavelengths of electromagnetic radiation at 635 nm are used in the determination of the particles diameter. The external laser source was also directed into the sample from above, rather than the side where the imaging laser is oriented, to further ensure that the external laser source would not artificially influence the particle size measurement. Additionally, control experiments were conducted to ensure that the external laser was not affecting the particle sizing measurements nor simply heating the entire aqueous sample.

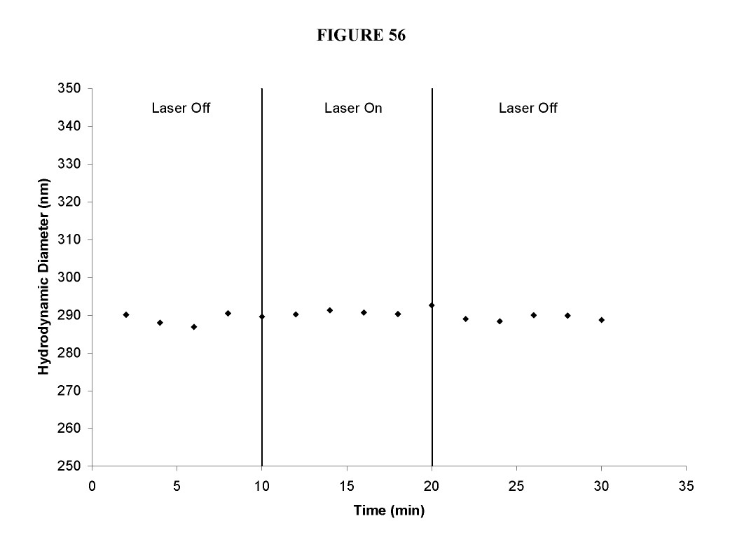

In the control experiments 3 ml of an aqueous suspension of blank IPN nanoparticles (i.e. particles containing no gold nanoparticle core) where placed in a quartz cuvette and loaded into the DLS instrument. An external laser source was also aligned above the cuvette to allow for even illumination of the entire aqueous suspension. The average hydrodynamic diameter of the particles was then measured continuously for 30 minutes with each individual average measurement taking 2 minutes to complete. For the first 10 minutes of this experiment the external 532 nm laser source was not activated then, at exactly 10 minutes, the laser was activated and irradiated the sample evenly for 10 minutes until it was again deactivated at exactly 20 minutes after the start of the experiment. Finally, measurements were collected for the remaining 10 minutes with the external laser source deactivated. The results of this experiment, shown in FIG. 56, clearly demonstrate that the external laser had no effect on the measured particle size of the blank IPN nanoparticles. The temperature of the aqueous suspension was also monitored through out the experiment using a digital needle point thermocouple and remained constant at 25.0±1.0° C.

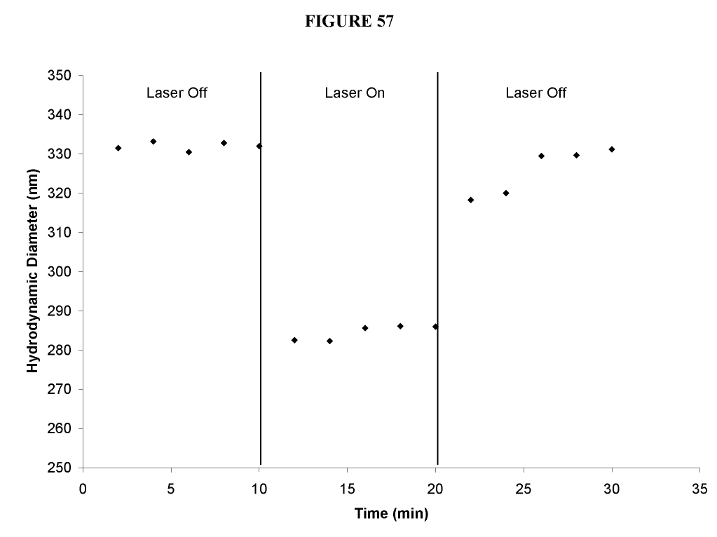

The response of the metal-polymer nanocomposite particles to an external laser source was also examined using the same method and setup described above for the blank IPN nanoparticles. The results of this experiment are shown in FIG. 57. It is clear that the external laser source is having an effect on the hydrodynamic diameter of the metal-polymer nanocomposite particles; however, based on the measured average hydrodynamic diameter of the system, it appears that the laser source is causing the particles to collapse rather than swell. In reality, the external laser source is driving the swelling of the metal-polymer nanocomposite particles. Unfortunately, the opposite trend is observed due to a combination of the limitations of the DLS instrument and the fact that only a portion of the IPN polymer particles have an encapsulated gold core.

Taking these two factors into account; the reason for the measured decrease in particle size is apparent. First, DLS relies on the scattering of light caused by the refractive index difference between the particles and their aqueous medium. As illustrated in FIGS. 48, 50, and 51, those nanocomposite particles which contained an encapsulated metal nanoparticle core were on average 50-100 nm in diameter larger than blank polymer nanoparticles which did not contain a metal nanoparticle core. Therefore, when sized together in the collapsed state these particles tended to exhibit an average particle diameter somewhere between 280-380 nm, typically centered around 330 nm diameter.

However, as hydrogel nanoparticles become swollen with water they become more and more transparent and do not scatter light as intensely. Since a large portion of the particles in the measured sample do not contain encapsulated metal nanoparticles they did not swell when the external laser source was turned on at the 10 minute mark and remained in the collapsed state. Therefore, when taking the average of the very large contribution of scattered light from the collapsed particles and the very small contribution from the swollen particles, the measured average was weighted heavily in favor of the smaller collapsed particles, in effect making the larger swollen metal-polymer nanocomposite particles invisible to the DLS instrument.

Because of this, when the laser light is turned on at the 10 minute time point the average diameter of the sample appears to instantaneously (within less than 2 minutes) drop to a value of approximately 285 nm diameter. This indicates that the metal-polymer nanocomposite particles are able to swell very rapidly as would be expected due to their small size and short characteristic diffusional length. Also, once the laser light is again turned off at the 20 minute time point, the particles are able to collapse back to their original size, and this is evident by the apparent increase in the average particle diameter of the sample driven by the increased scattering of the larger collapsed metal-polymer nanocomposite particles. The temperature of the aqueous suspension was again also monitored throughout the experiment using a digital needle point thermocouple and remained constant at 25.0±1.0° C.

Photoacoustic Imaging

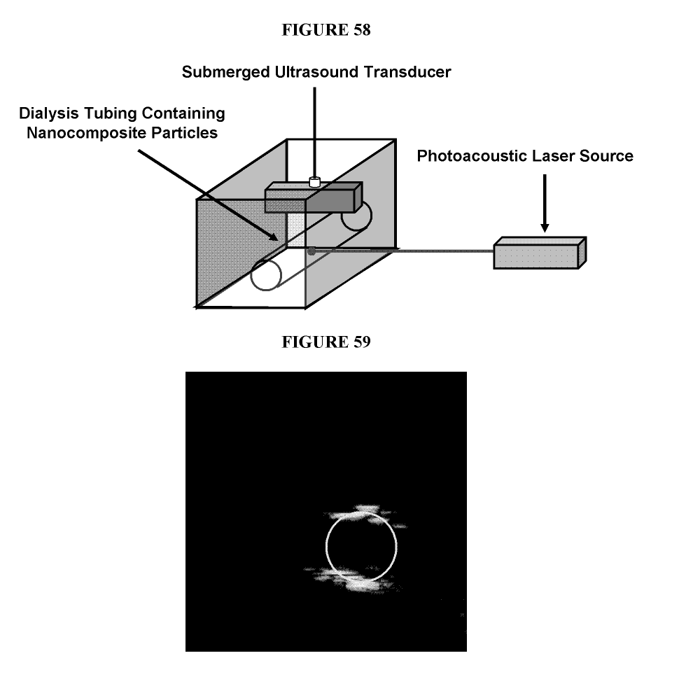

The final metal-polymer nanocomposite particles were also photoacoustically imaged utilizing the experimental setup schematically illustrated in FIG. 58. A standard ultrasound image of the dialysis tubing that was used to hold the nanocomposite particles during photoacoustic imaging was collected and is shown in FIG. 59. In this image, a yellow circle was added to illustrate the location of the dialysis tubing whose long axis is oriented into the plane of the image. The white area indicates the detected ultrasound signal that was produced by sound waves reflecting back to the transducer from the top and bottom of the dialysis tubing.

Control experiments were also conducted on a blank sample of pure ddH₂O to determine the amount of photoacoustic signal produced by the absorption of laser light by the dialysis tubing without the presence of nanocomposite particles. The results of this experiment are shown in the photoacoustic image in FIG. 60. As before, the dialysis tubing was oriented with its long axis into the plane of the image. Since a point source laser was used for imaging, only a small portion of the contents of the dialysis bag, that were in the optical path of the laser beam, were irradiated during an individual experiment. In this case, the laser source was directed into the bottom of the dialysis tubing from the right side with respect to the plane of the image. From this image, it is apparent that a small amount of the incident laser beam is absorbed by dialysis tubing and produces a small amount of photoacoustic signal as shown by the slight measured signal intensity.

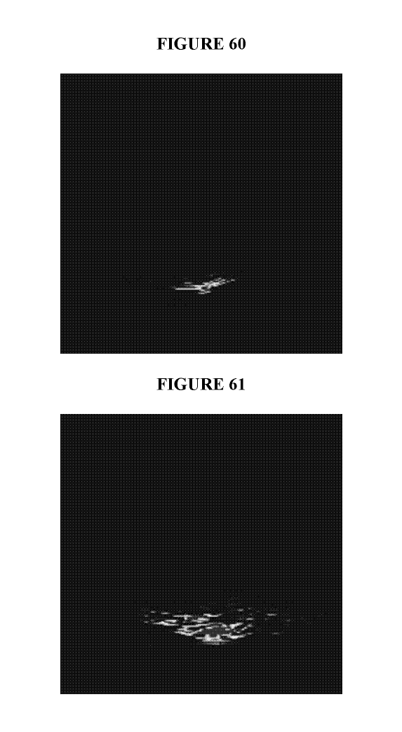

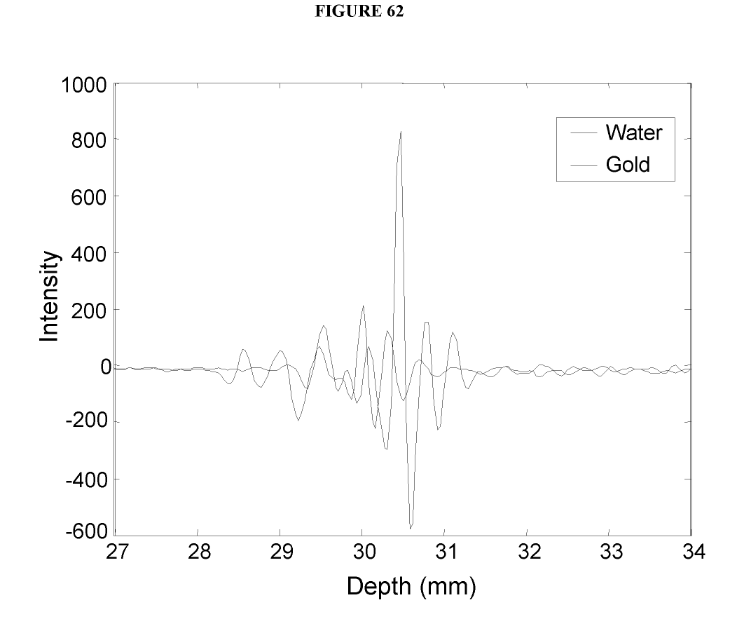

A sample of metal-polymer nanocomposite particles was photoacoustically imaged as well, using the same setup as described previously for the blank pure ddH₂O photoacoustic imaging experiment. The results of this experiment are shown in FIG. 61. As before, the dialysis tubing was oriented with its long axis into the plane of the image, and the laser source was directed into the bottom of the dialysis tubing from the right side. From FIG. 61 it is apparent that a much larger portion of the laser beam was absorbed leading to a greatly enhanced photoacoustic signal when compared to the blank signal. This enhanced signal production arises from the presence of the metal-polymer nanocomposite particles which absorb the 532 nm excitation laser source, to a much larger degree than the dialysis tubing alone, and dissipate that absorbed light energy in the form of broadband ultrasound waves and heat. The difference in signal intensity is also apparent in FIG. 62, which compares the photoacoustic signal intensity down the center of the dialysis tubing for both the blank ddH₂O sample and the metal-polymer nanocomposite sample.

In the above-example, metal-polymer nanocomposite particles comprised of a solid gold nanoparticle core and a thermally responsive poly(acrylamide)/poly(acrylic acid) interpenetrating polymer network (IPN) shell were successfully synthesized using an inverse microemulsion encapsulation technique. These metal-polymer nanocomposite particles were also surface functionalized with heterofunctional acryl-PEG-N-hydroxysuccinimide linear polymer chains using standard NHS chemistry to covalently bind to the primary amine groups of the polyacrylamide portion of the IPN shell. SEM imaging confirmed the spherical morphology of the nanocomposite particles. TEM and EDS analysis confirmed the successful encapsulation of gold nanoparticles within a portion of the as prepared nanocomposite particles. Zeta potential analysis was also used to confirm the successful covalent grafting of a charge shielding layer of linear PEG chains to the surface of the nanocomposite particles based on the shift in surface charge from a negative surface charge before PEGylation to a neutral or slightly positive surface charge after PEGylation.

The photothermally responsive swelling properties of these nanocomposite particles were examined using a dynamic light scattering instrument coupled with an external 532 nm laser excitation source. Although the results of this experiment showed a decrease in hydrodynamic diameter with laser activation, the opposite effect is actually occurring. When taking into account the limitations of the DLS instrument and the fact that only a portion of the IPN polymer particles have an encapsulated gold core, it is apparent that the laser is actually triggering the near instantaneous swelling of the polymer particles making them in effect invisible to the DLS instrument, thus resulting in a measured decrease in the average hydrodynamic diameter of the nanocomposite system. Finally, metal-polymer nanocomposite particles were also successfully excited using a 532 nm external laser source and subsequently imaged with a standard ultrasound transducer to produce a photoacoustic image of the nanocomposite particles.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A system comprising:
a thermally-active metal nanoshell; and
a temperature-responsive interpenetrating polymer network having at least one therapeutic agent disposed therein;
wherein the thermally-active metal nanoshell is proximate to the temperature-responsive interpenetrating polymer network.

2. The system of claim 1, wherein the metal nanoshell comprises a core comprising gold sulfide and a shell comprising gold.

3. The system of claim 1, wherein the interpenetrating polymer network further comprise attached PEG chains.

4. The system of claim 1, wherein the interpenetrating polymer network comprises two or more polymers chosen from poly(acrylic acid), polyacrylamide, any derivative thereof, and any combination thereof.

5. The system of claim 1, wherein the interpenetrating polymer network swells in response to an increase in temperature.

6. The system of claim 1, wherein the therapeutic agent is operable for being released upon heating the metal nanoshell.

7. The system of claim 1, further comprising a laser light source capable of emitting energy that is at least partially absorbed by the metal nanoshell.

8. The system of claim 1, further comprising a laser light source capable of emitting energy that is at least partially absorbed by the metal nanoshell and wherein the laser light source emits energy which has a wavelength of about 808 nanometers.

9. The system of claim 1, wherein the thermally-active metal nanoshell is disposed within at least a portion of the temperature-responsive interpenetrating polymer network.

10. A composition comprising a thermally-active metal nanoshell and a temperature-responsive interpenetrating polymer network.

11. The composition of claim 10, further comprising at least one therapeutic agent disposed within the interpenetrating polymer network.

12. The composition of claim 10, wherein the metal nanoshell comprises a core comprising gold sulfide and a shell comprising gold.

13. The composition of claim 10, wherein the interpenetrating polymer network further comprises attached PEG chains.

14. The composition of claim 10, wherein the interpenetrating polymer network comprises two or more polymers chosen from poly(acrylic acid), polyacrylamide, any derivative thereof, and any combination thereof.

15. The composition of claim 10, wherein the interpenetrating polymer network is capable of swelling in response to an increase in temperature.

16. The composition of claim 10, wherein the thermally-active metal nanoshell is disposed within at least a portion of the temperature-responsive interpenetrating polymer network.

17. A method comprising:
providing a plurality of particles according to claim 10; and
irradiating the particles so as to effect a temperature-induced swelling of the temperature-responsive interpenetrating polymer network.

18. The method of claim 17, further comprising releasing at least one therapeutic agent disposed within the interpenetrating polymer network.

19. The method of claim 17, wherein the interpenetrating polymer network comprises two or more polymers chosen from poly(acrylic acid), polyacrylamide, any derivative thereof, and any combination thereof.

20. The method of claim 17, wherein the metal nanoshell comprises a core comprising gold sulfide and a shell comprising gold

21. The method of claim 17, wherein the interpenetrating polymer network further comprise attached PEG chains.

22. The method of claim 17, wherein the step of irradiating the particles is performed by a laser light source capable of emitting energy that is at least partially absorbed by the metal nanoshell.

23. The method of claim 17, wherein the step of irradiating the particles is performed by a laser light source capable of emitting energy that is at least partially absorbed by the metal nanoshell and wherein the energy emitted from the laser has a wavelength of about 808 nanometers.
